(19) United States
(12) Patent Application Publication
(10) Pub. No.: US 2026/0021311 A1
(43) Pub. Date: Jan. 22, 2026

(54) METHOD AND APPARATUS FOR DETECTING OVERSENSING

(71) Applicant: Medtronic, Inc., Minneapolis, MN (US)

(72) Inventors: Yanina GRINBERG, Plymouth, MN (US); Keelia Marie ESCALANTE, Minneapolis, MN (US); Michelle Marie GALARNEAU, Eagan, MN (US); Vincent P. GANION, Blaine, MN (US); Todd J. SHELDON, North Oaks, MN (US); Paul R. SOLHEIM, Blaine, MN (US)

(21) Appl. No.: 18/869,256

(22) PCT Filed: May 26, 2023
(86) PCT No.: PCT/US2023/067552

§ 371 Date: Nov. 25, 2024

Related U.S. Application Data
(60) Provisional application No. 63/347,696, filed on Jun. 1, 2022.

(51) Int. Cl. A61N 1/37

(52) U.S. Cl. CPC A61N 1/3712

(57) ABSTRACT

A medical device includes a motion sensor configured to sense a motion signal. The medical device includes a control circuit configured to determine if the motion signal sensed over a motion metric time interval meets oversensing criteria when a cardiac electrical event signal is received during the motion metric time interval.

### RELATED APPLICATIONS

This application claims the benefit of provisional U.S. Patent Application No. 63/347,696, filed on Jun. 1, 2022, the entire content of which is incorporated herein by reference.

### TECHNICAL FIELD

This disclosure relates to a medical device configured to sense cardiac electrical event signals and detect oversensing.

### BACKGROUND

During normal sinus rhythm (NSR), the heartbeat is regulated by electrical signals produced by the sino-atrial (SA) node located in the right atrial wall. Each atrial depolarization signal produced by the SA node spreads across the atria, causing the depolarization and contraction of the atria, and arrives at the atrioventricular (AV) node. The AV node responds by propagating a ventricular depolarization signal through the bundle of His of the ventricular septum and thereafter to the bundle branches and the Purkinje muscle fibers of the right and left ventricles, sometimes referred to as the “His-Purkinje system.”

Patients with a conduction system abnormality, e.g., SA node dysfunction or poor AV node conduction, bundle branch block, or other conduction abnormalities, may receive a pacemaker to restore a more normal heart rhythm. A single chamber pacemaker coupled to a transvenous lead carrying electrodes positioned in the right atrium may provide atrial pacing to treat a patient having SA node dysfunction. When the AV node is functioning normally, single chamber atrial pacing may sufficiently correct the heart rhythm. The pacing-evoked atrial depolarizations may be conducted normally to the ventricles via the AV node and the His-Purkinje system maintaining normal AV synchrony. Some patients, however, may experience conduction abnormalities of the AV node, e.g., partial or complete AV block. AV block may be intermittent and may evolve over time. In the presence of high-degree AV block, atrial depolarizations may not be conducted to the ventricles on every atrial cycle or may be conducted but at a prolonged AV conduction time resulting in poor AV synchrony in the native heart rhythm. In this case, the patient may require a single chamber ventricular pacemaker or a dual chamber pacemaker.

A dual chamber pacemaker may be implanted in some patients to pace both the atrial and ventricular chambers and thereby maintain AV synchrony. The dual chamber pacemaker may be coupled to a transvenous atrial lead and a transvenous ventricular lead, for placing electrodes for sensing and pacing in both the atrial and ventricular chambers. The pacemaker itself is generally implanted in a subcutaneous pocket with the transvenous leads tunneled to the subcutaneous pocket. Intracardiac pacemakers have been introduced or proposed for implantation entirely within a patient's heart eliminating the need for transvenous leads. For example, an intracardiac pacemaker may provide sensing and pacing from within a heart chamber of a patient having a conduction abnormality to promote a more normal heart rhythm.

### SUMMARY

The techniques of this disclosure generally relate to a medical device configured to sense cardiac electrical event signals and identify oversensed event signals. The medical device may include a motion sensor for sensing a motion signal that includes ventricular mechanical event signals corresponding to ventricular contraction (and/or relaxation) following a ventricular electrical depolarization. The medical device may be configured to determine one or more features or metrics from the motion signal sensed during a specified, motion metric time interval. The medical device may use a motion metric for determining when a sensed ventricular event signal is an oversensed event signal that is falsely sensed as a ventricular event signal. The medical device may be a pacemaker configured to deliver ventricular pacing and may determine when, for example, cross-chamber atrial events, e.g., P-waves or atrial pacing pulses, or other noise signals are being oversensed by the medical device as false R-waves. In some examples, the medical device is a leadless pacemaker which may be implanted in a heart chamber for sensing cardiac signals and delivering ventricular pacing pulses.

In one example, the disclosure provides a medical device including a motion sensor configured to sense a motion signal and a control circuit configured to receive the motion signal sensed by the motion sensor during a motion metric time interval. The control circuit may be configured to receive a cardiac electrical event signal during the motion metric time interval and determine a motion metric from the motion signal sensed by the motion sensor during the motion metric time interval in response to the cardiac event signal being received during the motion metric time interval. The control circuit may be configured to determine that oversensing criteria are met based on at least the first motion metric and determine that the cardiac event signal is an oversensed signal in response to the oversensing criteria being met.

In another example, the disclosure provides a method including sensing a motion signal during a motion metric time interval, receiving a cardiac electrical event signal during the motion metric time interval and determining a motion metric from the motion signal sensed during the motion metric time interval in response to the cardiac event signal being received during the motion metric time interval. The method may further include determining that oversensing criteria are met based on at least the motion metric and determining that the cardiac event signal is an oversensed signal in response to the oversensing criteria being met.

In another example, the disclosure provides a non-transitory, computer-readable storage medium comprising a set of instructions which, when executed by a control circuit of a medical device, cause the medical device to sense a motion signal during a motion metric time interval, receive a cardiac electrical event signal during the motion metric time interval and determine a motion metric from the motion signal sensed during the motion metric time interval in response to the cardiac event signal being received during the motion metric time interval. The instructions may further cause the medical device to determine that oversensing criteria are met based on at least the motion metric and determine that the cardiac event signal is an oversensed signal in response to the oversensing criteria being met.

Further disclosed herein is the subject matter of the following clauses:

Clause 1. A medical device comprising a motion sensor configured to sense a motion signal and a control circuit. The control circuit may be configured to: receive the motion signal sensed by the motion sensor during a motion metric time interval, receive a cardiac electrical event signal during the motion metric time interval, determine a first motion metric from the motion signal sensed by the motion sensor during the motion metric time interval in response to the cardiac electrical event signal being received during the motion metric time interval, determine that oversensing criteria are met based on at least the first motion metric, and determine that the cardiac electrical event signal is an oversensed signal in response to the oversensing criteria being met.

Clause 2. The medical device of clause 1, further comprising a sensing circuit configured to sense a cardiac electrical signal and sense the cardiac electrical event from the cardiac electrical signal, and wherein the control circuit is further configured to receive the cardiac electrical event signal from the sensing circuit when the sensing circuit senses the cardiac electrical event.

Clause 3. The medical device of any of clauses 1-2 further comprising a pulse generator configured to a generate at least one pacing pulse in response to the control circuit determining that the cardiac electrical event signal is an oversensed signal.

Clause 4. The medical device of clause 3 wherein the pulse generator is further configured to generate the at least one pacing pulse by generating a plurality of pacing pulses at a fixed pacing rate.

Clause 5. The medical device of any of clauses 3-4 wherein the control circuit is further configured to start a pacing escape interval and withhold restarting the pacing escape interval in response to the cardiac electrical event signal being received during the motion metric time interval.

Clause 6. The medical device of clause 5 wherein the control circuit is further configured to determine that the pacing escape interval expires, and the pulse generator is further configured to generate a first pacing pulse of the at least one pacing pulse in response to the control circuit determining that the pacing escape interval is expired and the cardiac electrical event signal being an oversensed signal.

Clause 7. The medical device of clause 6 wherein the control circuit is further configured to determine that the pacing escape interval expires after the cardiac electrical event signal is received and before an expiration of the motion metric time interval and delay the first pacing pulse until at least an expiration of the motion metric time interval.

Clause 8. The medical device of any of clauses 3-7 wherein the control circuit is further configured to switch a pacing mode in response to determining that the cardiac electrical event signal is an oversensed signal.

Clause 9. The medical device of any of clauses 1-8 wherein the control circuit is further configured to adjust at least one cardiac event sensing control parameter in response to determining that the cardiac electrical event signal is an oversensed signal.

Clause 10. The medical device of clause 9 wherein the control circuit is further configured to adjust the at least one cardiac event sensing control parameter by adjusting a sensitivity setting.

Clause 11. The medical device of any of clauses 9-10 wherein the control circuit is further configured to adjust the at least one cardiac event sensing control parameter by switching to dual sensor cardiac event signal sensing using both a cardiac electrical signal and the motion signal for sensing the cardiac event signal.

Clause 12. The medical device of any of clauses 9-11 wherein the control circuit is further configured to adjust the at least one cardiac event sensing control parameter by adjusting a post-atrial ventricular blanking period.

Clause 13. The medical device of any of clauses 1-12, wherein the control circuit is further configured to determine the first motion metric by determining a summation of sample point amplitudes of the motion signal sensed during the motion metric time interval and determine that oversensing criteria are met based on at least the first motion metric by determining that the summation of sample point amplitudes is less than a threshold value.

Clause 14. The medical device of any of clauses 1-13 wherein the control circuit is further configured to determine the first motion metric by determining a count of sample point amplitudes of the motion signal sensed during the motion metric time interval that are greater than a threshold amplitude and determine that the oversensing criteria are met based on at least the first motion metric by determining that the count of sample point amplitudes is less than a threshold value.

Clause 15. The medical device of any of clauses 1-14 wherein the control circuit is further configured to determine the first motion metric based on an analysis of sample points during a first portion of the motion metric time interval, determine that the first motion metric is greater than a threshold value, determine a second motion metric based on an analysis of sample points during a second portion of the motion metric time interval that is different than the first portion of the motion metric time interval and determine that the oversensing criteria are met based on at least the second motion metric when the first motion metric is greater than the threshold value.

Clause 16. The medical device of any of clauses 1-14 wherein the control circuit is further configured to determine the first motion metric based on an analysis of sample points during a first portion of the motion metric time interval, determine that the first motion metric is greater than a threshold value, determine a second motion metric based on an analysis of sample points during a second portion of the motion metric time interval that is different than the first portion of the motion metric time interval and determine that the motion signal is unreliable for determining that the received cardiac electrical event signal is an oversensed event based on at least the second motion metric when the first motion metric is greater than the threshold value.

Clause 17. The medical device of clause 16 further comprising a pulse generator configured to generate at least one pacing pulse in response to the control circuit determining that the motion signal is unreliable for determining that the received cardiac electrical event signal is an oversensed event.

Clause 18. The medical device of any of clauses 1-17 wherein the control circuit is further configured to set the motion metric time interval to a multiple of a pacing interval.

Clause 19. The medical device of any of clauses 1-18 wherein the control circuit is further configured to start the motion metric time interval independent of a starting time of a cardiac cycle.

Clause 20. The medical device of any of clauses 1-19, wherein the control circuit is further configured to determine the first motion metric upon expiration of the motion metric time interval.

Clause 21. A method comprising sensing a motion signal during a motion metric time interval, receiving a cardiac electrical event signal during the motion metric time interval, determining a first motion metric from the motion signal sensed during the motion metric time interval in response to the cardiac electrical event signal being received during the motion metric time interval, determining that oversensing criteria are met based on at least the first motion metric and determining that the cardiac electrical event signal is an oversensed signal in response to the oversensing criteria being met.

Clause 22. The method of clause 21 further comprising sensing a cardiac electrical signal and sensing the cardiac electrical event from the cardiac electrical signal.

Clause 23. The method of any of clauses 21-22 further comprising generating at least one pacing pulse in response to determining that the cardiac electrical event signal is an oversensed signal.

Clause 24. The method of clause 23 further comprising generating the at least one pacing pulse by generating a plurality of pacing pulses at a fixed pacing rate.

Clause 25. The method of any of clauses 23-24 further comprising starting a pacing escape interval and withholding restarting the pacing escape interval in response to the cardiac electrical event signal being received during the motion metric time interval.

Clause 26. The method of clause 25 further comprising determining that the pacing escape interval expires and generating a first pacing pulse of the at least one pacing pulse in response to determining that the pacing escape interval is expired and the cardiac electrical event signal being an oversensed signal.

Clause 27. The method of clause 26 further comprising determining that the pacing escape interval expires after the cardiac electrical event signal is received and before an expiration of the motion metric time interval and delaying the first pacing pulse until at least an expiration of the motion metric time interval.

Clause 28. The method of any of clauses 22-27 further comprising switching a pacing mode in response to determining that the cardiac electrical event signal is an oversensed signal.

Clause 29. The method of any of clauses 21-28 further comprising adjusting at least one cardiac event sensing control parameter in response to determining that the cardiac electrical event signal is an oversensed signal.

Clause 30. The method of clause 29 further comprising adjusting the at least one cardiac event sensing control parameter by adjusting a sensitivity setting.

Clause 31. The method of any of clauses 29-30 further comprising adjusting the at least one cardiac event sensing control parameter by switching to dual sensor cardiac event signal sensing using both a cardiac electrical signal and the motion signal for sensing cardiac event signals.

Clause 32. The method of any of clauses 29-31 further comprising adjusting the at least one cardiac event sensing control parameter by adjusting a post-atrial ventricular blanking period.

Clause 33. The method of any of clauses 21-32 further comprising determining the first motion metric by determining a summation of sample point amplitudes of the motion signal sensed during the motion metric time interval and determining that oversensing criteria are met based on at least the first motion metric by determining that the summation of sample point amplitudes is less than a threshold value.

Clause 34. The method of any of clauses 21-33 further comprising determining the first motion metric by determining a count of sample point amplitudes of the motion signal sensed during the motion metric time interval that are greater than a threshold amplitude and determining that the oversensing criteria are met based on at least the first motion metric by determining that the count of sample point amplitudes is less than a threshold value.

Clause 35. The method of any of clauses 21-34 further comprising determining the first motion metric based on an analysis of sample points during a first portion of the motion metric time interval, determining that the first motion metric is greater than a threshold value, determining a second motion metric based on an analysis of sample points during a second portion of the motion metric time interval that is different than the first portion of the motion metric time interval and determining that the oversensing criteria are met based on at least the second motion metric when the first motion metric is greater than the threshold value.

Clause 36. The method of any of clauses 21-34 further comprising determining the first motion metric based on an analysis of sample points during a first portion of the motion metric time interval, determining that the first motion metric is greater than a threshold value, determining a second motion metric based on an analysis of sample points during a second portion of the motion metric time interval that is different than the first portion of the motion metric time interval and determining that the motion signal is unreliable for determining that the received cardiac electrical event signal is an oversensed event based on at least the second motion metric when the first motion metric is greater than the threshold value.

Clause 37. The method of clause 36 further comprising generating at least one pacing pulse in response to determining that the motion signal is unreliable for determining that the received cardiac electrical event signal is an oversensed event.

Clause 38. The method of any of clauses 21-37 further comprising setting the motion metric time interval to a multiple of a pacing interval.

Clause 39. The method of any of clauses 21-38 further comprising starting the motion metric time interval independent of a starting time of a cardiac cycle.

Clause 40. The method of any of clauses 21-39 further comprising determining the first motion metric upon expiration of the motion metric time interval.

Clause 41. A non-transitory, computer-readable storage medium storing a set of instructions which, when executed by a control circuit of a medical device, cause the medical device to sense a motion signal during a motion metric time interval, receive a cardiac electrical event signal during the motion metric time interval, determine a first motion metric from the motion signal sensed during the motion metric time interval in response to the cardiac electrical event signal being received during the motion metric time interval, determine that oversensing criteria are met based on at least the first motion metric and determine that the cardiac electrical event signal is an oversensed signal in response to the oversensing criteria being met.

Clause 42. The non-transitory, computer-readable storage medium of clause 41 further comprising instructions that cause the medical device to sense a cardiac electrical signal and sense the cardiac electrical event from the cardiac electrical signal.

Clause 43. The non-transitory, computer-readable storage medium of clauses 41-42 further comprising instructions that cause the medical device to generate at least one pacing pulse in response to determining that the cardiac electrical event signal is an oversensed signal.

44. The non-transitory, computer-readable storage medium of clause 43 further comprising instructions that cause the medical device to generate the at least one pacing pulse by generating a plurality of pacing pulses at a fixed pacing rate.

Clause 45. The non-transitory, computer-readable storage medium of any of clauses 43-44 further comprising instructions that cause the medical device to start a pacing escape interval and withhold restarting the pacing escape interval in response to the cardiac electrical event signal being received during the motion metric time interval.

Clause 46. The non-transitory, computer-readable storage medium of clause 45 further comprising instructions that cause the medical device to determine that the pacing escape interval expires and generate a first pacing pulse of the at least one pacing pulse in response to determining that the pacing escape interval is expired and the cardiac electrical event signal being an oversensed signal.

Clause 47. The non-transitory, computer-readable storage medium of clause 46 further comprising instructions that cause the medical device to determine that the pacing escape interval expires after the cardiac electrical event signal is received and before an expiration of the motion metric time interval and delay the first pacing pulse until at least an expiration of the motion metric time interval.

Clause 48. The non-transitory, computer-readable storage medium of clauses 42-47 further comprising instructions that cause the medical device to switch a pacing mode in response to determining that the cardiac electrical event signal is an oversensed signal.

Clause 49. The non-transitory, computer-readable storage medium of any of clauses 41-48 further comprising instructions that cause the medical device to adjust at least one cardiac event sensing control parameter in response to determining that the cardiac electrical event signal is an oversensed signal.

Clause 50. The non-transitory, computer-readable storage medium of clause 49 further comprising instructions that cause the medical device to adjust the at least one cardiac event sensing control parameter by adjusting a sensitivity setting.

Clause 51. The non-transitory, computer-readable storage medium of any of clauses 49-50 further comprising instructions that cause the medical device to adjust the at least one cardiac event sensing control parameter by switching to dual sensor cardiac event signal sensing using both a cardiac electrical signal and the motion signal for sensing cardiac event signals.

Clause 52. The non-transitory, computer-readable storage medium of any of clauses 49-51 further comprising instructions that cause the medical device to adjust the at least one cardiac event sensing control parameter by adjusting a post-atrial ventricular blanking period.

Clause 53. The non-transitory, computer-readable storage medium of any of clauses 41-52 further comprising instructions that cause the medical device to determine the first motion metric by determining a summation of sample point amplitudes of the motion signal sensed during the motion metric time interval and determine that oversensing criteria are met based on at least the first motion metric by determining that the summation of sample point amplitudes is less than a threshold value.

Clause 54. The non-transitory, computer-readable storage medium of any of clauses 42-53 further comprising instructions that cause the medical device to determine the first motion metric by determining a count of sample point amplitudes of the motion signal sensed during the motion metric time interval that are greater than a threshold amplitude and determine that the oversensing criteria are met based on at least the first motion metric by determining that the count of sample point amplitudes is less than a threshold value.

Clause 55. The non-transitory, computer-readable storage medium of any of clauses 41-54 further comprising instructions that cause the medical device to determine the first motion metric based on an analysis of sample points during a first portion of the motion metric time interval, determine that the first motion metric is greater than a threshold value, determine a second motion metric based on an analysis of sample points during a second portion of the motion metric time interval that is different than the first portion of the motion metric time interval and determine that the oversensing criteria are met based on at least the second motion metric when the first motion metric is greater than the threshold value.

Clause 56. The non-transitory, computer-readable storage medium of any of clauses 41-54 further comprising instructions that cause the medical device to determine the first motion metric based on an analysis of sample points during a first portion of the motion metric time interval, determine that the first motion metric is greater than a threshold value, determine a second motion metric based on an analysis of sample points during a second portion of the motion metric time interval that is different than the first portion of the motion metric time interval and determine that the motion signal is unreliable for determining that the received cardiac electrical event signal is an oversensed event based on at least the second motion metric when the first motion metric is greater than the threshold value.

Clause 57. The non-transitory, computer-readable storage medium of clause 56 further comprising instructions that cause the medical device to generate at least one pacing pulse in response to determining that the motion signal is unreliable for determining that the received cardiac electrical event signal is an oversensed event.

Clause 58. The non-transitory, computer-readable storage medium of any of clauses 41-57 further comprising instructions that cause the medical device to set the motion metric time interval to a multiple of a pacing interval.

Clause 59. The non-transitory, computer-readable storage medium of any of clauses 41-58 further comprising instructions that cause the medical device to start the motion metric time interval independent of a starting time of a cardiac cycle.

Clause 60. The non-transitory, computer-readable storage medium of any of clauses 41-59 further comprising instructions that cause the medical device to determine the first motion metric upon expiration of the motion metric time interval.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating an implantable medical device (IMD) system that may be used to sense cardiac signals and provide cardiac pacing.

FIG. 2 is a conceptual diagram of the ventricular pacemaker shown in FIG. 1 according to one example.

FIG. 3 is conceptual diagram of the pacemaker of FIG. 1 shown implanted in an alternative location for sensing cardiac signals and delivering ventricular pacing according to another example.

FIG. 4 is a conceptual diagram of a ventricular pacemaker according to another example.

FIG. 5 is a conceptual diagram of an example configuration of the ventricular pacemaker of FIG. 1.

FIG. 6 is a diagram of a motion signal and cardiac events occurring over several cardiac cycles depicting operations that may be performed by a ventricular pacemaker according to some examples.

FIG. 7 is a diagram of a motion signal and cardiac events occurring over several cardiac cycles depicting operations that may be performed by a ventricular pacemaker according to some examples.

FIG. 8 is a flow chart of a method for detecting oversensing by processing circuitry of a pacemaker according to some examples.

FIG. 9 is a flow chart of a method for detecting oversensing by processing circuitry of a pacemaker according to some examples.

FIG. 10 is a flow chart of a method for detecting and responding to oversensing according to another example.

### DETAILED DESCRIPTION

In general, this disclosure describes a medical device and method for determining when an event signal sensed from a cardiac electrical signal is oversensed as a false cardiac event signal, e.g., an intrinsic ventricular R-wave. An oversensed event signal, which may be an atrial P-wave, a T-wave, or a non-cardiac noise signal such as electromagnetic interference, may be identified based on processing and analysis of a motion signal sensed by the medical device. As described below, a motion signal, e.g., an accelerometer signal, may be produced by a motion sensor included in a cardiac device implanted in or on a heart chamber. The motion signal may include cardiac event signals attendant to the mechanical activity of the heart, e.g., heart chamber contraction and relaxation. For example, ventricular mechanical event signals corresponding to ventricular contraction and/or closure of the atrioventricular valves caused by ventricular contraction may be present in a motion signal sensed within an atrial or ventricular heart chamber.

A cardiac pacemaker that is configured to deliver ventricular pacing pulses may be configured to sense ventricular electrical events, e.g., intrinsic R-waves attendant to ventricular depolarization. The pacemaker may control the delivery of ventricular pacing pulses based on R-wave sensing. For example, when an R-wave is sensed, a scheduled ventricular pacing pulse may be withheld. A ventricular pacing interval may be restarted in response to the sensed R-wave. In the absence of a sensed intrinsic R-wave, a scheduled ventricular pacing pulse is delivered upon expiration of a pacing interval, which may be an AV pacing interval started in response to an atrial event or the ventricular pacing interval started in response to a preceding sensed R-wave or ventricular pacing pulse. In some instances, an atrial electrical event, e.g., an intrinsic P-wave attendant to atrial depolarization or a delivered atrial pacing pulse, can be oversensed as an intrinsic R-wave. Sensing of an atrial electrical event as a false R-wave is referred to herein as “cross-chamber oversensing.” Cross-chamber oversensing (CCOS) of atrial events as false R-waves may cause the cardiac pacemaker to withhold a ventricular pacing pulse when a ventricular pacing pulse may actually be needed. In some cases, other electrical signals may be falsely sensed as intrinsic R-waves, such as electromagnetic interference (EMI) or other electrical noise in the cardiac electrical signal.

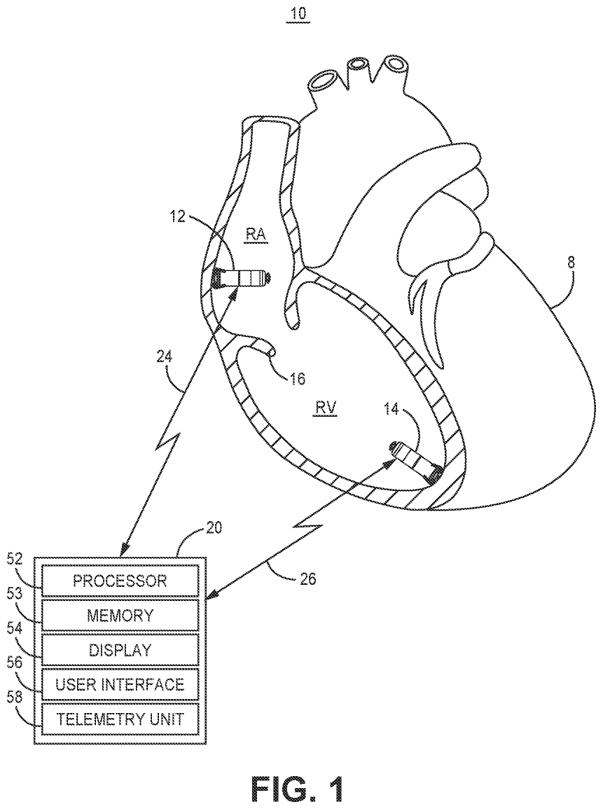

FIG. 1 is a conceptual diagram illustrating an implantable medical device (IMD) system 10 that may be used to sense cardiac signals and provide cardiac pacing. IMD system 10 is shown including an atrial pacemaker 12, implanted within the right atrium (RA), and a ventricular pacemaker 14, shown implanted in the right ventricular (RV) of a patient's heart 8. IMD system 10 may include both the atrial pacemaker 12 and the ventricular pacemaker 14 for providing dual chamber pacing therapies.

In some examples, pacemakers 12 and 14 are transcatheter leadless pacemakers that can be implanted wholly within a heart chamber. Pacemakers 12 and 14 may be reduced in size compared to subcutaneously implanted pacemakers and may be generally cylindrical in shape to enable transvenous implantation via a delivery catheter. Pacemakers 12 and 14 may each be a leadless pacemaker and include housing-based electrodes for sensing cardiac electrical signals and delivering pacing pulses.

Atrial pacemaker 12 may be wholly implanted within the right atrium (RA) and may be implanted along the lateral endocardial wall as shown in FIG. 1 though other locations are possible within or on the RA, different than the location shown. Ventricular pacemaker 14 may be wholly implanted within the right ventricle (RV) as shown or on a ventricular chamber, e.g., at an epicardial location. Ventricular pacemaker 14 is shown positioned in the RV along an endocardial wall, e.g., near the RV apex though other locations are possible. For example, ventricular pacemaker 14 may be positioned in or on the LV and configured to sense cardiac signals and deliver ventricular pacing to the LV. The techniques disclosed herein are not limited to the pacemaker locations shown in the example of FIG. 1 and a variety of atrial and ventricular implant locations of atrial pacemaker 12 and ventricular pacemaker 14, respectively: may be possible for sensing and pacing an atrial chamber and a ventricular chamber in a dual pacemaker system. However, in some examples, atrial pacemaker 12 may be excluded. Some patients may not require atrial pacing when SA node function is intact.

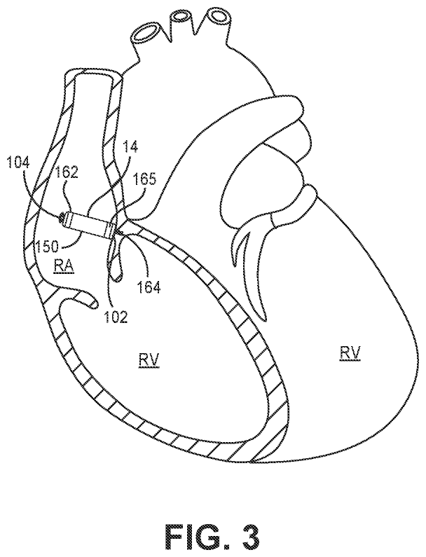

In other examples, ventricular pacemaker 14 may be configured for implantation in the RA for sensing atrial signals and delivering ventricular pacing pulses via the native His-Purkinje conduction system from a right atrial approach, e.g., as described below in conjunction with FIG. 3. The techniques disclosed herein are not necessarily limited to a particular ventricular pacemaker location and may be practiced in a ventricular pacemaker implanted in a variety of operative locations for providing cardiac signal sensing and cardiac pacing to at least a ventricular chamber.

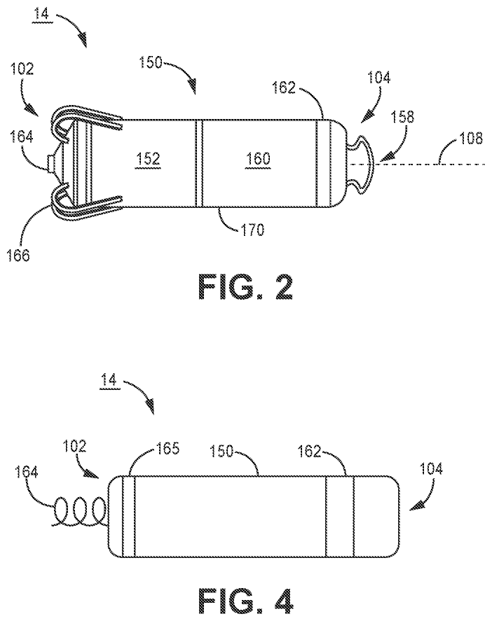

Atrial pacemaker 12 may include cardiac electrical signal sensing circuitry configured to sense atrial P-waves attendant to the depolarization of the atrial myocardium and a pulse generator for generating and delivering an atrial pacing pulse in the absence of a sensed intrinsic atrial P-wave. The atrial electrical signals may be sensed using the housing based electrodes that are also used to deliver pacing pulses to the RA. As generally described below in conjunction with FIG. 2 with regard to ventricular pacemaker 14, atrial pacemaker 12 may have a tip electrode located on a distal end of atrial pacemaker 12 that is placed against or in close proximity to atrial tissue. The tip electrode may function as a cathode electrode for pacing and sensing functions. Atrial pacemaker 12 may include a ring electrode that circumscribes the cylindrical housing of pacemaker 12 for functioning as an anode electrode for pacing and sensing functions. Atrial pacemaker 12 may have two (or more) housing based electrodes that may take the form of any of a helix, fishhook, button, hemispherical, segmented, ring or other type of electrode or combination of electrodes for providing a pair of atrial pacing and sensing electrodes. Atrial pacemaker 12 may include one or more fixation members, e.g., fixation tines, a fixation helix, or other fixation members for engaging with atrial tissue to anchor pacemaker 12 at an atrial implant site.

In other examples, an atrial pacemaker may be implanted in an implant pocket, e.g., subcutaneously or submuscularly, and coupled to a lead extending into the right atrium and carrying electrodes for sensing and pacing in the right atrium. A single chamber pacemaker connected to an atrial lead may be substituted for the leadless atrial pacemaker 12 in a dual pacemaker system that includes a ventricular pacemaker operating according to the techniques disclosed herein. The atrial pacemaker connected to the atrial lead can be co-implanted with the leadless ventricular pacemaker 14 implanted for delivering ventricular pacing for providing dual chamber pacing in a two device system.

Ventricular pacemaker 14 includes cardiac electrical signal sensing circuitry configured to sense intrinsic ventricular R-waves attendant to ventricular myocardial depolarizations and a pulse generator for generating and delivering a ventricular pacing pulse in the absence of a sensed R-wave. The ventricular electrical signals may be sensed using the housing based electrodes that are also used to deliver ventricular pacing pulses.

Pacemaker 14 may be configured to control the delivery of ventricular pacing pulses in a non-atrial tracking ventricular pacing mode, also referred to herein as an “asynchronous pacing mode,” during which ventricular pacing pulses may be scheduled at ventricular lower rate interval (LRI), which may at times be a temporary LRI set to a rate smoothing interval or a rate response interval, e.g., during an episode of increased patient physical activity. Examples of asynchronous pacing modes can be denoted as VVI(R) or VDI(R) pacing modes, during which ventricular pacing is delivered, with either single chamber ventricular sensing or dual chamber atrial and ventricular sensing. A scheduled ventricular pacing pulse is inhibited when an intrinsic ventricular event signal, e.g., an R-wave, is sensed. At times, ventricular pacemaker 14 may operate in an asynchronous pacing mode without sensing ventricular or atrial event signals, which may be denoted as a VOO pacing mode. In other instances, ventricular pacemaker 14 may operate in an asynchronous ventricular triggered pacing mode, which may be denoted as a VVT pacing mode.

Ventricular pacemaker 14 may also be configured to operate in an atrial synchronous pacing mode to promote synchrony between atrial activation and ventricular activation, e.g., by delivering ventricular pacing pulses at an atrioventricular (AV) interval following sensed atrial event signals. The atrial synchronous ventricular pacing mode may be denoted as a VDD pacing mode. Atrial contraction producing the active ventricular filling phase can be sensed from a cardiac motion signal by ventricular pacemaker 14 as an atrial event signal, also referred to herein as an “A4 event.” The motion signal may be sensed from a motion sensor, such as an accelerometer, enclosed by the housing of ventricular pacemaker 14. The motion signal produced by the accelerometer includes motion (e.g., acceleration) signals caused by ventricular and atrial mechanical events. For example, acceleration of blood flowing from the RA into the RV through the tricuspid valve 16 between the RA and RV caused by atrial systole, and referred to as the “atrial kick,” may be sensed by ventricular pacemaker 14 as an A4 signal from the acceleration signal produced by an accelerometer included in ventricular pacemaker 14.

Atrial P-waves that are attendant to atrial depolarization are relatively low amplitude signals in the near-field ventricular cardiac electrical signal received by ventricular pacemaker 14 (e.g., compared to the near-field R-wave) and therefore can be difficult to reliably detect from the cardiac electrical signal acquired by ventricular pacemaker 14 when implanted in or on a ventricular chamber. Atrial-synchronized ventricular pacing by ventricular pacemaker 14 or other functions that rely on atrial event signal sensing may rely at least in part on sensing atrial event signals from a motion signal produced by a motion sensor implemented in the ventricular pacemaker 14 in some examples.

In other examples, an atrial pacemaker 12, or other intracardiac or extracardiac device capable of sensing P-waves, that is configured to transmit a communication signal or trigger signal indicating the timing of an intrinsic atrial P-wave or a delivered atrial pacing pulse may be included in the IMD system 10. Ventricular pacemaker 14 may be configured to receive the communication signal from atrial pacemaker 12 and, in response to the communication signal, deliver an atrial synchronous ventricular pacing pulse. At times, ventricular pacemaker 14 may oversense a P-wave as a false R-wave prior to receiving the transmitted communication signal, which may interfere with the ventricular pacing function of ventricular pacemaker 14.

When an R-wave is sensed by the ventricular pacemaker 14, a scheduled ventricular pacing pulse may be withheld or inhibited. When atrial pacemaker 12 delivers an atrial pacing pulse, the atrial pacing pulse may be oversensed as a false R-wave by ventricular pacemaker 14. At other times, depending on the implant location of pacemaker 14 relative to the atrial chambers, the P-wave signal may be large enough in the cardiac electrical signal sensed by ventricular pacemaker 14 to be oversensed as a false R-wave. When this cross-chamber oversensing occurs, ventricular pacemaker 14 may withhold a ventricular pacing pulse, e.g., by restarting an LRI, in response to the oversensed atrial event signal. According to the techniques disclosed herein, oversensed event signals are detected based on a motion metric determined from the motion sensor signal sensed during a specified motion metric time interval. As described below with the accompanying drawings, a motion metric may be determined from sample points spanning the motion metric time interval in some examples. The motion metric may be representative of the total motion during the time interval that is sensed by the motion sensor in some examples. When ventricular pacemaker 14 senses a ventricular event signal from a sensed cardiac electrical signal during the motion metric time interval, processing circuitry may compare the motion metric determined from the motion signal sensed during the motion metric time interval to a threshold value or other oversensing criteria. If the motion metric meets oversensing criteria, e.g., if a motion metric representative of motion sensed during the motion metric time interval is less than the threshold, the sensed ventricular event signal may be determined to be an oversensed event signal, e.g., a cross-chamber oversensed event.

Pacemakers 12 and 14 may be capable of bidirectional wireless communication with an external device 20 for programming sensing and pacing control parameters, which may include control parameters used for sensing the motion signal, determining one or more motion signal metrics, sensing cardiac event signals, and providing pacing therapy. Aspects of external device 20 may generally correspond to the external programming/monitoring unit disclosed in U.S. Pat. No. 5,507,782 (Kieval, et al.), hereby incorporated herein by reference in its entirety. External device 20 is often referred to as a “programmer” because it is typically used by a physician, technician, nurse, clinician or other qualified user for programming operating parameters in an implantable medical device, e.g., pacemaker 12 or pacemaker 14. External device 20 may be located in a clinic, hospital or other medical facility. External device 20 may alternatively be embodied as a home monitor or a handheld device that may be used in a medical facility, in the patient's home, or another location. Operating parameters, including sensing and therapy delivery control parameters, may be programmed into pacemaker 12 or pacemaker 14 by a user interacting with external device 20.

External device 20 may include a processor 52, memory 53, display unit 54, user interface 56 and telemetry unit 58. Processor 52 controls external device operations and processes data and signals received from pacemaker 12 or 14. Display unit 54 may generate a display, which may include a graphical user interface, of data and information relating to pacemaker functions to a user for reviewing pacemaker operation and programmed parameters as well as cardiac electrical signals, motion signals, data derived therefrom or other physiological data that may be acquired by atrial pacemaker 12 and/or ventricular pacemaker 14 and transmitted to external device 20 during an interrogation session. For example, ventricular pacemaker 14 may generate an output for transmission to external device 20 including any one or more of motion signal metrics, pacing and sensing event histories, device operating parameters and device diagnostic data.

Transmitted data may include an episode of a cardiac electrical signal produced by pacemaker sensing circuitry including markers indicating pacing pulse delivery and sensed cardiac event signals, e.g., ventricular sensed event signals and/or atrial sensed event signals and any delivered ventricular pacing pulses. Transmitted data may include a cardiac electrical signal episode recorded by ventricular pacemaker 14 in response to identifying an oversensed event signal. The display unit 54 may display the cardiac electrical signal episode with annotated sensed event signals, pacing pulse markers, and/or identified oversensed event signals. The transmitted data may include a response taken by ventricular pacemaker 14 based on identifying one or more oversensed event signals. The response may include a ventricular pacing response and/or a ventricular sensing response as further described in various examples given below.

User interface 56 may include a mouse, touch screen, keypad or the like to enable a user to interact with external device 20 to initiate a telemetry session with pacemaker 12 or pacemaker 14 for retrieving data from and/or transmitting data to the pacemaker 12 or 14, including programmable parameters for controlling sensing and pacing functions.

Telemetry unit 58 includes a transceiver and antenna configured for bidirectional communication with a telemetry circuit included in respective pacemakers 12 and 14 and is configured to operate in conjunction with processor 52 for sending and receiving data relating to pacemaker functions via communication link 24 and communication link 26, respectively.

Telemetry unit 58 may establish a wireless bidirectional communication link 24 or 26 with pacemaker 12 or 14, respectively. Communication link 24 may be established using a radio frequency (RF) link such as BLUETOOTH®, Wi-Fi, Medical Implant Communication Service (MICS) or other communication bandwidth. In some examples, external device 20 may include a programming head that is placed proximate pacemaker 12 or 14 to establish and maintain a communication link 24 or 26, and in other examples external device 20 and pacemakers 12 and 14 may be configured to communicate using a distance telemetry algorithm and circuitry that does not require the use of a programming head and does not require user intervention to maintain a communication link.

It is contemplated that pacemakers 12 and 14 may be configured to communicate with each other and/or external device 20 wirelessly using a radiofrequency communication protocol or using tissue conduction communication or other wireless communication means. In some examples, atrial pacemaker 12 may transmit a signal to ventricular pacemaker 14, for example, to trigger ventricular pacemaker 14 to deliver a ventricular pacing pulse.

It is further contemplated that external device 20 may be in wired or wireless connection to a communications network via a telemetry circuit that includes a transceiver and antenna or via a hardwired communication line for transferring data to a centralized database or computer to allow remote management of the patient. Remote patient management systems including a centralized patient database may enable a clinician to view data relating to sensing and pacing functions performed by pacemaker 12 and/or pacemaker 14.

FIG. 2 is a conceptual diagram of the ventricular pacemaker 14 shown in FIG. 1 according to one example. Ventricular pacemaker 14 includes a housing 150 having a distal end 102 and a proximal end 104. The lateral sidewall 170 of housing 150 may be generally cylindrical extending from distal end 102 to proximal end 104. In other examples, housing 150 may have a generally prismatic shape. Ventricular pacemaker 14 includes electrodes 162 and 164 spaced apart along the housing 150 of pacemaker 14 for sensing cardiac electrical signals and delivering pacing pulses. Electrode 164 is shown as a tip electrode extending from a distal end 102 of pacemaker 14. Electrode 162 is shown as a ring electrode circumscribing the lateral sidewall 170 of housing 150, for example adjacent proximal end 104. Distal end 102 is referred to as “distal” in that it is expected to be the leading end as pacemaker 14 is advanced through a delivery tool, such as a catheter, and placed against a targeted pacing site.

Electrodes 162 and 164 form an anode and cathode pair for bipolar cardiac pacing and sensing. In alternative embodiments, ventricular pacemaker 14 may include two or more ring electrodes, two tip electrodes, and/or other types of electrodes exposed along housing 150 for delivering electrical stimulation to heart 8 and sensing cardiac electrical signals. Electrodes 162 and 164 may be, without limitation, titanium, platinum, iridium or alloys thereof and may include a low polarizing coating, such as titanium nitride, iridium oxide, ruthenium oxide, platinum black, among others. Electrodes 162 and 164 may be positioned at locations along ventricular pacemaker 14 other than the locations shown.

Housing 150 is formed from a biocompatible material, such as a stainless steel or titanium alloy. In some examples, the housing 150 may include an insulating coating. Examples of insulating coatings include parylene, urethane, PEEK, or polyimide, among others. The entirety of the housing 150 may be insulated, but only electrodes 162 and 164 uninsulated. Electrode 164 may serve as a cathode electrode and can be coupled to internal circuitry, e.g., a pacing pulse generator and cardiac electrical signal sensing circuitry. enclosed by housing 150 via an electrical feedthrough crossing housing 150. Electrode 162 may be formed as a conductive portion of housing 150 defining a ring electrode that is electrically isolated from the other portions of the housing 150 as generally shown in FIG. 2. In other examples, the entire periphery of the housing 150 may function as an electrode that is electrically isolated from tip electrode 164, instead of providing a localized ring electrode such as electrode 162. Electrode 162 formed along an electrically conductive portion of housing 150 serves as a return anode during pacing and sensing.

The housing 150 may include a control electronics subassembly 152 and a battery subassembly 160, which provides power to the control electronics subassembly 152. Control electronics subassembly 152 houses the electronics for sensing cardiac signals, producing pacing pulses and controlling therapy delivery and other functions of pacemaker 14 as described herein. According to the techniques disclosed herein, ventricular pacemaker 14 includes a mechanical cardiac signal sensor that produces a signal responsive to cardiac motion. In the illustrative examples disclosed herein, the sensor is a motion sensor such as an accelerometer responsive to acceleration forces and is enclosed by the housing of pacemaker 14. In other examples, the mechanical sensor may be a pressure sensor, a flow sensor, an acoustical sensor, an impedance sensor or other sensor responsive to cardiac motion. The mechanical cardiac signal sensor provides a signal correlated to cardiac mechanical events, e.g., acceleration due to heart chamber contraction and relaxation, to a processor included in control electronics subassembly 152 for signal processing and analysis for determining a motion metric that can be used by the processor for identifying oversensed event signals, e.g., CCOS event signals. The mechanical sensor signal may further be used for sensing atrial systolic event signals for controlling atrial synchronous ventricular pacing in some examples.

An accelerometer implemented as the cardiac mechanical signal sensor may be a multi-axis or multi-dimensional accelerometer where each axis of the accelerometer generates an acceleration signal in a different dimension. In a multi-dimensional accelerometer, the sensor elements may be arranged orthogonally with each sensor element axis orthogonal relative to the other sensor element axes. In some examples, the accelerometer may have one “longitudinal” axis that is parallel to or aligned with the longitudinal axis 108 of pacemaker 14 and two orthogonal axes that extend in radial directions relative to the longitudinal axis 108. Practice of the techniques disclosed herein, however, are not limited to a particular orientation of the accelerometer within or along housing 150 or a particular number of axes. Orthogonal arrangement of the elements of a multi-axis accelerometer is not necessarily required. A one-dimensional accelerometer may be used to obtain a motion signal from which a motion metric may be determined for detecting CCOS (or oversensing of other event signals in the cardiac electrical signal). In other examples, a two dimensional accelerometer or other multi-dimensional accelerometer may be used.

Each axis of a single or multi-dimensional accelerometer may be defined by a piezoelectric element, micro-electrical mechanical system (MEMS) device or other sensor element capable of producing an electrical signal in response to changes in acceleration imparted on the sensor element, e.g., by converting the acceleration to a force or displacement that is converted to the electrical signal. Each sensor element or axis may produce an acceleration signal corresponding to a vector aligned with the axis of the sensor element. A vector signal of a multi-dimensional accelerometer (also referred to herein as a “multi-axis” accelerometer) for use in sensing cardiac mechanical events or determining motion metrics may be selected as a single axis signal or a combination of two or more axis signals. For example, one, two or all three axis signals produced by a three dimensional accelerometer may be selected for processing and analysis for use in sensing atrial event signals and/or determining a motion metric used for detecting oversensing.

Ventricular pacemaker 14 may include features for facilitating deployment to and fixation at an implant site. For example, pacemaker 14 may include a set of fixation tines 166 to secure pacemaker 14 to cardiac tissue, e.g., by actively engaging with the endocardium and/or interacting with the ventricular trabeculae. Fixation tines 166 are configured to anchor pacemaker 14 to position electrode 164 in operative proximity to a targeted pacing site for delivering therapeutic electrical stimulation pulses. Numerous types of active and/or passive fixation members may be employed for anchoring or stabilizing pacemaker 14 in an implant position.

Ventricular pacemaker 14 may optionally include a delivery tool interface 158. Delivery tool interface 158 may be located at the proximal end 104 of ventricular pacemaker 14 and is configured to connect to a delivery device, such as a catheter, used to position pacemaker 14 at an implant location during an implantation procedure, for example within the right ventricle. Features of ventricular pacemaker 14 described in conjunction with FIG. 2 may be analogous to features of atrial pacemaker 12, e.g., fixation tines 166, delivery tool interface 158, and tip electrode 164 and ring electrode 162 all carried by a housing 150 enclosing a power supply and electronics. It is recognized, however, that the geometry, size, shape, materials, arrangement and other aspects of such analogous features may be altered and adapted as needed for optimizing atrial sensing and pacing functions and/or accommodating an atrial implant location.

FIG. 3 is a conceptual diagram of the pacemaker 14 of FIG. 1 shown implanted in an alternative location for sensing cardiac signals and delivering ventricular pacing according to another example. In this example, ventricular pacemaker 14 is implanted in the RA for providing ventricular pacing from an atrial location. In some cases, ventricular pacemaker 14 may be positioned for delivering ventricular pacing pulses via the heart's native conduction system and/or ventricular myocardium. The distal end 102 of ventricular pacemaker 14 may be positioned at the inferior end of the interatrial septum, beneath the AV node and near the tricuspid valve annulus to position tip electrode 164 for advancement into the interatrial septum toward the His bundle of the native His-Purkinje conduction system. Ring electrode 162 spaced proximally from tip electrode 164 may be used as the return electrode with the cathode tip electrode 164 for pacing the right and left ventricles via the His-Purkinje system. Tip electrode 164 may be positioned to capture at least a portion of the His bundle and/or ventricular myocardium for delivering ventricular pacing from an atrial implant location of pacemaker 14.

Ventricular pacemaker 14 may be capable of dual chamber sensing and pacing in some examples. For instance, a distal ring electrode 165 may be included on pacemaker housing 150 and can be used in combination with the proximal ring electrode 162 for sensing atrial P-waves and, in some examples, delivering atrial pacing pulses. When tip electrode 164 and ring electrode 162 are used for sensing ventricular R-waves from a RA implant location, P-waves may be oversensed as false R-waves. Ventricular pacemaker 14 may be configured to detect and respond to this CCOS according to the techniques disclosed herein. Examples of various pacing electrode arrangements and medical device configurations for providing cardiac pacing along the native conduction system of the heart, which may be combined with the oversensing detection and response techniques disclosed herein, are generally disclosed in U.S. Publication No. 2019/0083779, granted as U.S. Pat. No. 11,426,578, (Yang, et al.) and U.S. Pat. No. 11,007,369 (Sheldon, et al.), the entire content of all of which is incorporated herein by reference in its entirety.

FIG. 4 is a conceptual diagram of ventricular pacemaker 14 according to another example. In FIG. 2, the cathode tip electrode 164 is shown as a button or hemispherical type electrode that may contact the ventricular tissue when distal end 102 is anchored at an implant site by fixation tines 166 within or on a ventricular chamber. In the example of FIG. 4, the cathode tip electrode 164 is shown as a screw-in helical electrode which may provide fixation of ventricular pacemaker 14 at an implant site as well as serving as a pacing and sensing electrode. In the example of FIG. 4, ventricular pacemaker 14 may be configured to provide ventricular pacing from a right atrial approach when ventricular pacemaker 14 is implanted in the RA. Electrode 164 can be advanced from within the right atrial chamber to a His bundle pacing location or a ventricular septal pacing location.

In this case, tip electrode 164 and return anode electrode 162 may be used for pacing the ventricles, e.g., via the His bundle or another portion of the His-Purkinje conduction system and/or ventricular myocardium. A second ring electrode 165 may be provided at or near distal end 102 for use as a second cathode electrode for providing atrial pacing and sensing in combination with the return anode 162 in some examples. Ventricular pacemaker 14 may include two or more electrodes which may be ring electrodes, helical electrodes, hook electrodes, button electrodes, hemispherical electrodes or other types of electrodes arranged along housing 150 for providing at least ventricular electrical signal sensing (e.g., R-wave sensing) and ventricular pacing, e.g., by capturing at least a portion of the His-Purkinje conduction system and/or surrounding ventricular myocardial tissue, in some examples.

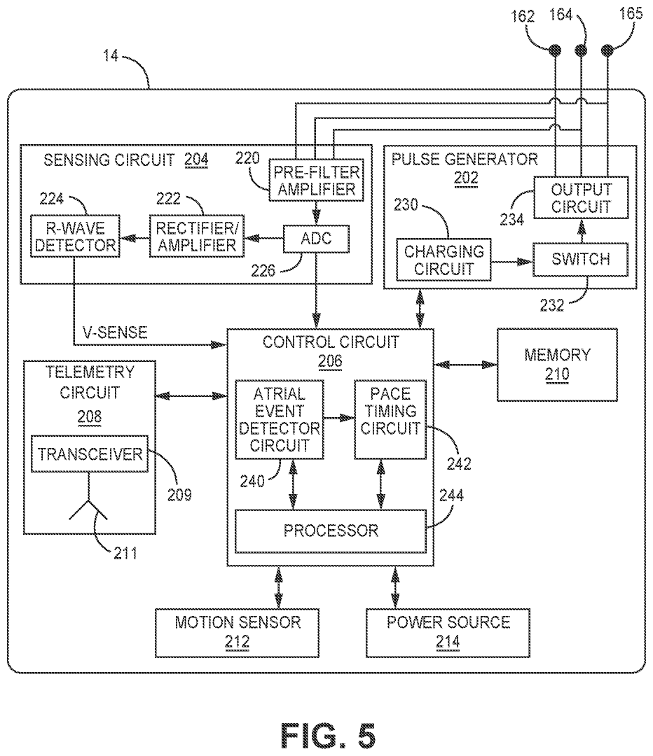

FIG. 5 is a conceptual diagram of an example configuration of ventricular pacemaker 14 according to some examples. The circuitry and components and the associated functionality described in conjunction with FIG. 5 may be incorporated in ventricular pacemaker 14 as shown FIG. 1 for implantation in a ventricular location or as shown in FIG. 3 for implantation in an atrial location. Ventricular pacemaker 14 includes a pulse generator 202, a cardiac electrical signal sensing circuit 204, a control circuit 206, telemetry circuit 208, memory 210, motion sensor 212 and a power source 214. The various circuits represented in FIG. 5 may be combined on one or more integrated circuit boards which include a specific integrated circuit (ASIC), an electronic circuit, a processor (shared, dedicated, or group) and memory that execute one or more software or firmware programs, a combinational logic circuit, state machine or other suitable components that provide the described functionality.

Motion sensor 212 includes an accelerometer in the examples described herein. Motion sensor 212 is not limited to being an accelerometer, however, and other sensors of mechanical function or motion of the heart may be utilized successfully in pacemaker 14 for sensing a signal responsive to cardiac motion and identifying oversensed event signals according to the techniques described herein. Other sensors that could be implemented in ventricular pacemaker 14 include a pressure sensor, impedance sensor, acoustical sensor, or flow sensor, as examples that produce a signal responsive to motion, e.g., contraction and relaxation, of the heart chambers and/or the associated opening and closing of heart valves. Examples of motion sensors that may be implemented in motion sensor 212 when implemented as an accelerometer include piezoelectric sensors and MEMS devices.

Motion sensor 212 may be enclosed by the housing 150 (shown in FIGS. 2 and 4). As described above, motion sensor 212 may include a multi-axis sensor, e.g., a two-dimensional or three-dimensional accelerometer, with each axis providing an axis signal that may be analyzed individually or in combination for determining a motion metric and, at least in some examples, sensing atrial event signals for tracking during an atrial synchronous ventricular pacing mode. Motion sensor 212 produces an electrical signal correlated to motion or vibration of sensor 212 (and pacemaker 14), e.g., when subjected to flowing blood and cardiac motion. The motion sensor 212 may include one or more filter, amplifier, rectifier, analog-to-digital converter (ADC) and/or other components for producing a motion signal that is passed to control circuit 206. For example, each vector signal produced by each individual axis of a multi-axis accelerometer may be filtered by a high pass filter, e.g., a 10 Hz high pass filter, or a bandpass filter, e.g., a 10 Hz to 35 Hz bandpass filter. The high pass filter may be raised (e.g., to 15 Hz) if needed to pass ventricular event signals that may have higher frequency content. In some examples, high pass filtering is performed with no low pass filtering. In other examples, each accelerometer axis signal is filtered by a low pass filter, e.g., a 30 to 35 Hz low pass filter, with or without high pass filtering. It is to be understood that the filtering cutoff frequencies applied to the motion signal may be selected based on the cardiac event signal properties in the motion signal and other factors and the filter examples provided here are not intended to be limiting.

The filtered signal may be digitized by an ADC and rectified by a rectifier included in motion sensor 212. The filtered and rectified signal may be passed to control circuit 206 for use by processing circuitry of control circuit 206 for determining a motion metric and detecting oversensing according to the techniques disclosed herein. Control circuit 206 may include an atrial event detector circuit 240 for sensing atrial event signals from a motion signal received from motion sensor 212. For example, when ventricular pacemaker 14 is implanted in the RV, atrial synchronous pacing may be controlled based on the timing of atrial event signals sensed from the motion signal. The motion signal received by atrial event detector circuit 240 from motion sensor 212 may be filtered differently than a motion signal received by control circuit 206 for determining a motion metric that is used in detecting oversensing of false ventricular event signals, e.g., CCOS of atrial P-waves or atrial pacing pulses. Furthermore the motion sensor axis or combination of axes used for obtaining a motion signal provided to atrial event detector circuit 240 may be the same or different than the axis or combination of axes used for determining the motion metric for detecting oversensing of false ventricular event signals.

Furthermore, in some examples, control circuit 206 may be configured to determine a patient physical activity metric correlated to the level of physical exertion and metabolic demand of the patient from a signal received from motion sensor 212. The patient activity metric may be used by control circuit 206 to control rate response pacing. In some examples, the patient activity metric and the motion metric used for detecting oversensing of false ventricular event signals may be the same metric. When the patient physical activity metric increases, ventricular pacemaker 14 may switch to an asynchronous pacing mode to provide ventricular rate support needed by the patient during increased physical activity.

Control circuit 206 may be configured to determine a sensor indicated pacing rate (SIR) from the patient physical activity metric for controlling rate response pacing. In some examples, the accelerometer axis signal(s) used for determining a patient activity metric may be filtered differently than the axis signal(s) used for determining a motion metric for detecting oversensing of false ventricular event signals and/or differently than filtering applied to the axis signal(s) used for sensing atrial event signals for providing atrial synchronous ventricular pacing. For example, motion sensor 212 may include a low pass filter having an upper cutoff frequency of 10 Hz for passing a low pass filtered patient activity signal to processor 244 for determining a patient activity metric. Motion sensor 212 may include a bandpass filter having a lower cutoff frequency of 10 Hz or higher and an upper cutoff frequency of 25 Hz to 40 Hz for passing a bandpass filtered cardiac motion signal from one or more of the accelerometer axes to control circuit 206 for determining a motion metric for detecting oversensing and/or for detecting atrial event signals during atrial synchronous ventricular pacing. In other examples, the same signal used for determining a patient activity metric may be used by control circuit 206 for determining a motion metric for detecting oversensing of false ventricular event signals.

The patient activity metric may be determined by control circuit 206 at a desired frequency, e.g., every two seconds, for use in determining an SIR that meets the metabolic requirements of the patient based on physical activity. The SIR may vary between the programmed minimum lower rate during periods of rest (minimal activity metric) and a maximum upper pacing rate during periods of maximum exertion. The SIR may be determined according to an SIR transfer function, which may include different rates of change of the SIR over different ranges of the patient activity metric.

In some examples, the activity metric is determined as an activity count. In these instances, control circuit 206 includes a counter that may track the activity count as the number of times the patient activity signal from motion sensor 212 crosses a threshold amplitude during an activity count interval, for example a 2-second interval. The count at the end of each activity count interval is correlated to patient body motion during the activity count interval. Example methods for obtaining an activity count over an n-second interval are generally disclosed in U.S. Pat. No. 5,720,769 (van Oort), incorporated herein by reference in its entirety.

In other examples, an activity metric may be obtained from the patient physical activity signal by integrating or summing activity signal sample points over an activity count interval, e.g., a two-second interval, though longer or shorter intervals of time may be used for determining a patient activity metric. The activity metric may be converted to a target heart rate to meet the patient's metabolic demand. The target heart rate may be converted to an SIR based on an SIR transfer function that includes a lower rate set point and an activities of daily living (ADL) range and a maximum upper rate. Examples of methods for establishing an SIR transfer function applied to patient activity metrics determined from an intracardiac motion signal are generally disclosed in U.S. Pat. No. 9,724,518 (Sheldon, et al.), incorporated herein by reference in its entirety.

Control circuit 206 may use the patient physical activity metric as a motion metric for detecting oversensing of false ventricular event signals in some examples. When the activity metric is less than a threshold value at the end of the two-second interval (or another selected time interval) and a ventricular event is sensed by sensing circuit 204 during the two-second interval, control circuit 206 may determine that the sensed ventricular event signal is an oversensed event signal. In other examples, a different motion metric may be determined using a different time interval, different filtering properties of the accelerometer axis signal(s), and/or different axis signal(s) than the time interval, filtering properties and axis signal(s) used for determining the patient physical activity metric used in controlling rate response pacing.

The ventricular mechanical event signals in a motion signal received from motion sensor 212 are expected to be relatively high amplitude or even the highest amplitude event signals in the motion signal. The presence or absence of relatively large ventricular mechanical event signals in the motion signal during a motion metric time interval as indicated based on at least one determined motion metric may be determined by control circuit 206 for detecting oversensing of a false ventricular event signal during the motion metric time interval.

One example of an accelerometer for use in implantable medical devices that may be implemented in conjunction with the techniques disclosed herein is generally disclosed in U.S. Pat. No. 5.885,471 (Ruben, et al.), incorporated herein by reference in its entirety. An implantable medical device arrangement including a piezoelectric accelerometer for detecting patient motion is disclosed, for example, in U.S. Pat. No. 4,485,813 (Anderson, et al.) and U.S. Pat. No. 5,052,388 (Sivula, et al.), both of which patents are hereby incorporated by reference herein in their entirety. Examples of three-dimensional accelerometers that may be implemented in ventricular pacemaker 14 and used for determining motion metrics and detecting oversensing of false ventricular event signals using the presently disclosed techniques are generally described in U.S. Pat. No. 5,593,431 (Sheldon) and U.S. Pat. No. 6,044,297 (Sheldon), both of which are incorporated herein by reference in their entirety. Other accelerometer designs may be used for producing an electrical signal that is correlated to motion imparted on pacemaker 14 due to cardiac mechanical events.

Sensing circuit 204 is configured to receive at least one cardiac electrical signal via electrodes coupled to ventricular pacemaker 14, e.g., via tip electrode 164 and ring electrode 162. When ring electrode 165 is present, as in the example of ventricular pacemaker 14 shown in FIG. 4, a second cardiac electrical signal may be received via electrodes 165 and 162, for example. As such, sensing circuit 204 may have multiple sensing channels, e.g., a ventricular sensing channel and an atrial sensing channel.

A raw cardiac electrical signal for sensing ventricular electrical event signals, e.g., R-waves, is received from electrodes 164 and 162 (or 164 and 165) by a pre-filter and amplifier circuit 220. Pre-filter and amplifier circuit 220 may include a high pass filter to remove DC offset, e.g., a 2.5 to 5 Hz high pass filter, or a wideband filter having a bandpass of 2.5 Hz, to 100 Hz or narrower to remove DC offset and high frequency noise. Pre-filter and amplifier circuit 220 may further include an amplifier to amplify the raw cardiac electrical signal passed to analog-to-digital converter (ADC) 226. ADC 226 may pass a multi-bit, digital electrogram (EGM) signal to control circuit 206 for storage in memory 210 and/or further analysis. For example, an episode of the EGM signal passed to control circuit 206 from ADC 226 may be stored in memory 210 in response to oversensing detection in some examples, to facilitate troubleshooting and corrective actions taken to reduce the likelihood of future oversensing of false ventricular event signals.

The digital signal from ADC 226 may be passed to rectifier and amplifier circuit 222, which may include a rectifier, bandpass filter, and amplifier for passing a cardiac signal to cardiac event detector 224. Cardiac event detector 224 (shown as an R-wave detector) may include a sense amplifier or other detection circuitry that compares the incoming rectified, cardiac electrical signal to a cardiac event sensing threshold, which may be an auto-adjusting threshold. For example, when the incoming signal crosses an R-wave sensing threshold, the cardiac event detector 224 produces a ventricular sensed event signal (Vsense) that is passed to control circuit 206. In various examples, cardiac event detector circuit 224 may receive the digital output of ADC 226 for sensing R-waves by a comparator, morphological signal analysis of the digital EGM signal or other R-wave detection techniques.

Processor 244 may provide sensing control signals to sensing circuit 204, e.g., R-wave sensing threshold adjustment parameters, ventricular sensitivity, and various blanking and refractory intervals applied to the cardiac electrical signal for controlling R-wave sensing such as a post-atrial ventricular blanking period. R-wave sensing threshold adjustment parameters may be used by sensing circuit 204 for setting the R-wave sensing threshold amplitude to a starting value, which may be a specified percentage (e.g., 50% to 80%) of a maximum peak amplitude of the EGM signal during a post-ventricular blanking period following an R-wave sensing threshold crossing by the ventricular EGM signal. Sensing circuit 204 may adjust the R-wave sensing threshold from the starting value toward the ventricular sensitivity according to one or more decay rates (over corresponding decay intervals) and/or one or more step drops in amplitude (upon expiration of corresponding drop time intervals). The ventricular sensitivity defines the minimum EGM signal amplitude that can be sensed as an R-wave.

Vsense signals passed from cardiac event detector 224 to control circuit 206 may be used for scheduling ventricular pacing pulses by pace timing circuit 242 at an LRI, which may be a base pacing rate interval, a temporary rate response interval or a rate smoothing interval. Vsense signals may be used by atrial event detector circuit 240 for use in setting post-ventricular blanking and sensing windows for detecting atrial mechanical event signals (e.g., A4 signals) from a motion signal received from motion sensor 212. In response to receiving a Vsense signal from sensing circuit 204, control circuit 206 may compare a motion metric determined from a motion signal received from motion sensor 212 to oversensing criteria as further described below.

In some examples, cardiac event detector 224 of sensing circuit 204 may be configured to sense P-waves from an atrial electrical signal, e.g., sensed by electrodes 162 and 165. P-waves may be sensed based on a P-wave sensing threshold crossing. Control circuit 206 may receive atrial sensed event signals from sensing circuit 204 for setting an AV pacing interval used to control atrial synchronous ventricular pacing pulse delivery, e.g., when pacemaker 14 is implanted in the right atrium. When sensing circuit 204 is configured to receive an atrial electrical signal and a ventricular electrical signal, components included in an atrial sensing channel and in a ventricular sensing channel of sensing circuit 204 may be separate or shared between both sensing channels in various examples. For example, pre-filter/amplifier 220 and/or ADC 226 may be shared by both an atrial sensing channel and a ventricular sensing channel with separate outputs being passed to a P-wave detector of an atrial sensing channel and to an R-wave detector of the ventricular sensing channel. Different filtering and amplification may be applied to the output of ADC 226 before passing the separate signals to the respective P-wave detector and R-wave detector.

Control circuit 206 may include atrial event detector circuit 240, pace timing circuit 242, and processor 244. Control circuit 206 may receive Vsense signals (and in some examples atrial sensed event signals) from sensing circuit 204 for use in controlling ventricular pacing therapies. When sensing circuit 204 is configured to sense P-waves and pass atrial sensed event signals to control circuit 206, pace timing circuit 242 may start an AV pacing interval in response to the atrial sensed event signal from sensing circuit 204 to control the timing of atrial synchronous ventricular pacing pulses. In other examples, as indicated above, atrial event detector circuit 240 may be configured to sense atrial event signals from a signal received from motion sensor 212. Techniques for sensing atrial event signals from a motion signal and controlling atrial synchronized ventricular pacing pulses are generally disclosed in U.S. Pat. No. 10,449,366 (Splett, et al.), incorporated herein by reference in its entirety.

In some examples, atrial event detector circuit 240 receives a motion signal from motion sensor 212 and may start a post-ventricular atrial blanking period in response to a ventricular pacing pulse delivered by pulse generator 202 or a Vsense signal from sensing circuit 204. Pace timing circuit 242 may start a ventricular LRI, which may be set to a base pacing rate interval or a temporary LRI or a rate smoothing interval in response to the ventricular pacing pulse delivered or the Vsense signal. Upon expiration of the post-ventricular atrial blanking period, atrial event detector circuit 240 may start a ventricular diastolic event window during which a first, high atrial event sensing threshold amplitude is applied to the motion signal. If the motion signal crosses the first, high atrial event sensing threshold amplitude, control circuit 206 senses the atrial event signal and triggers a ventricular pacing pulse at an AV pacing interval while operating in an atrial synchronous ventricular pacing mode. Pace timing circuit 242 may restart the ventricular LRI in response to the delivered ventricular pacing pulse.

If the ventricular diastolic event window expires without sensing the atrial event signal, atrial event detector circuit 240 may apply a second, low atrial event sensing threshold amplitude to the motion signal starting from the expiration of the ventricular diastolic event window. If the motion signal crosses the low atrial event sensing threshold amplitude, control circuit 206 senses the atrial event signal and triggers a ventricular pacing pulse delivered by pulse generator 202 at an AV pacing interval during atrial synchronous ventricular pacing operations. Pace timing circuit 242 may restart the ventricular LRI in response to the delivered ventricular pacing pulse.

If an atrial event signal is not sensed by atrial event detector circuit 240 prior to the expiration of a ventricular LRI, pulse generator 202 may deliver an asynchronous pacing pulse and restart the LRI. When a Vsense signal is received by control circuit 206 prior to an atrial event signal being sensed by atrial event detector circuit 240 or prior to an AV pacing interval or an LRI expiring, the scheduled ventricular pacing pulse may be inhibited, and the LRI may be restarted. In some examples, the scheduled ventricular pacing pulse may be delayed as a pending pacing pulse until the Vsense signal is verified or determined to be an oversensed event signal based on analysis of the motion signal. According to the techniques disclosed herein, in response to a Vsense signal occurring during a motion metric time interval, control circuit 206 may compare a subsequently determined motion metric to oversensing criteria. If the motion metric meets the oversensing criteria, control circuit 206 may determine that the Vsense signal is an oversensed event signal. Control circuit 206 may perform a ventricular pacing response to the oversensed event signal by withholding restarting of a running ventricular pacing interval and/or may schedule a ventricular pacing pulse to avoid ventricular asystole. The motion metric is correlated to the amount of motion sensed by motion sensor 212 during the motion metric time interval. The oversensing criteria may include one or more thresholds, ranges or other values that are applied to at least one metric or feature determined from the motion signal during a specified motion metric time interval for distinguishing a time interval that includes a ventricular mechanical event signal, e.g., as evidence of ventricular systolic contraction, from a time interval that does not include a ventricular mechanical event signal.

For example, when a motion metric is less than an oversensing threshold indicating that a ventricular mechanical event signal is unlikely to be present in the motion signal sensed over the motion metric time interval during which a Vsense signal is received from sensing circuit 204, control circuit 206 may determine that the Vsense signal is an oversensed event signal. Control circuit 206 may perform a ventricular pacing response and/or perform a ventricular sensing response based on the determination of an oversensed event signal. For example, instead of inhibiting a scheduled ventricular pacing pulse and restarting a ventricular pacing interval in response to the Vsense signal, the Vsense signal identified as an oversensed event signal may be ignored for the purposes of resetting a ventricular pacing interval in some examples. As described below; control circuit 206 may allow any pace timing intervals and/or R-wave sensing related timing intervals currently running to continue running without being canceled or restarted.

For instance, an LRI, a rate smoothing interval, a rate response interval, a post-ventricular atrial blanking period, a ventricular diastolic event window, an R-wave sensing threshold decay time or any other timing interval used to control R-wave sensing and/or ventricular pacing that was started prior the oversensed event signal may continue running without interruption and without being restarted. If an atrial event is subsequently sensed after the oversensed event signal, e.g., a P-wave by sensing circuit 204 or an A4 event signal by atrial event detector circuit 240, pacing timing circuit 242 may start the AV pacing interval for scheduling the atrial synchronous ventricular pacing pulse. If a ventricular pacing interval set to an LRI, rate smoothing interval or a rate response interval that was started prior to the oversensed event signal expires, a ventricular pacing pulse may be delivered by pulse generator 202. Other examples of ventricular pacing responses and ventricular sensing responses that may be performed by control circuit 206 in response to an identified oversensed event signal are described below. The oversensed event signal detection may be logged in memory 210 and an episode of an EGM signal including the oversensed event signal may be stored in memory 210 for transmission to external device 20.

Pace timing circuit 242 may receive atrial sensed event signals from cardiac event detector 224 of sensing circuit 204 for use in controlling the timing of ventricular pacing pulses delivered by pulse generator 202, e.g., via electrodes 162 and 164, during atrial synchronous ventricular pacing, e.g., when ventricular pacemaker 14 is implanted in the right atrium. Additionally or alternatively, pace timing circuit 242 may receive atrial sensed event signals, for triggering an atrial synchronous ventricular pacing pulse, from atrial event detector circuit 240 of control circuit 206, e.g., when pacemaker 14 is implanted in a right ventricular location and atrial event signals are sensed from a motion signal received from motion sensor 212. In still other examples, pace timing circuit 242 may start an AV interval or trigger pulse generator 202 to deliver a ventricular pacing pulse in response to pacemaker 14 receiving a communication signal from another device, e.g., atrial pacemaker 12, indicating the timing of an atrial P-wave or delivered atrial pacing pulse. Processor 244 may include one or more clocks for generating clock signals that are used by pace timing circuit 242 to time out the AV pacing interval and/or an LRI (or other ventricular pacing interval) for providing ventricular pacing according to an operating pacing mode.

Patients being implanted with ventricular pacemaker 14, e.g., as shown in FIG. 1 or FIG. 3, will typically have at least some degree of AV conduction block such that an atrial synchronous ventricular pacing pulse can be delivered when an atrial P-wave is not conducted to the ventricles (or is conducted but with a long AV conduction delay). Ventricular pacemaker 14 may be configured to schedule a ventricular pacing pulse at a ventricular pacing interval from a preceding ventricular event, either a ventricular pacing pulse or a Vsense signal. At times, an atrial event signal may not be sensed during an atrial synchronous pacing mode or the atrial rate may be faster than a maximum atrial tracking rate. In these instances, pulse generator 202 may deliver an asynchronous ventricular pacing pulse in response to the ventricular pacing interval expiring. At other times, the ventricular pacemaker 14 may be operating in an asynchronous ventricular pacing mode. e.g., a VDI or VVI pacing mode, which may be a rate response pacing mode to provide rate support to the patient during an episode of increased physical activity.

Pulse generator 202 generates electrical pacing pulses that are delivered to pace the ventricles of the patient's heart via cathode electrode 164 and return anode electrode 162. In examples including atrial pacing capabilities, e.g., when pacemaker 14 is implanted in the right atrium, pulse generator 202 may generate electrical pacing pulses for pacing the atria, e.g., using electrodes 165 and 162. In addition to providing control signals to pace timing circuit 242 and pulse generator 202 for controlling the timing of pacing pulses, processor 244 may retrieve programmable pacing control parameters, such as pacing pulse amplitude and pacing pulse width, which are passed to pulse generator 202 for controlling pacing pulse delivery.

Pulse generator 202 may include charging circuit 230, switching circuit 232 and an output circuit 234. Charging circuit 230 is configured to receive current from power source 214 and may include a holding capacitor that may be charged to a pacing pulse amplitude under the control of a voltage regulator included in charging circuit 230. The pacing pulse amplitude may be set based on a control signal from control circuit 206. Switching circuit 232 may control when the holding capacitor of charging circuit 230 is coupled to the output circuit 234 for delivering the pacing pulse. For example, switching circuit 232 may include a switch that is activated by a timing signal received from pace timing circuit 242 upon expiration of a pacing escape interval and kept closed for a programmed pacing pulse width to enable discharging of the holding capacitor of charging circuit 230. The holding capacitor, previously charged to the pacing pulse voltage amplitude, can be discharged across electrodes 162 and 164 (or other available pacing electrode vector) through an output capacitor of output circuit 234 for the programmed pacing pulse duration. Examples of pacing circuitry generally disclosed in U.S. Pat. No. 5,507,782 (Kieval, et al.) and in U.S. Pat. No. 8,532,785 (Crutchfield, et al.), both of which patents are incorporated herein by reference in their entirety, may be implemented in pacemaker 14 for charging a pacing capacitor to a predetermined pacing pulse amplitude under the control of control circuit 206 and delivering a pacing pulse.

Memory 210 may include computer-readable instructions that, when executed by control circuit 206, cause control circuit 206 to perform various functions attributed throughout this disclosure to pacemaker 14. The computer-readable instructions may be encoded within memory 210. Memory 210 may include any non-transitory, computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or other digital media with the sole exception being a transitory propagating signal.

Memory 210 may store sensed event data based on Vsense signals received from sensing circuit 204 and may flag Vsense signals as false Vsense signals when identified as oversensed event signals based on motion metrics. In some examples, memory 210 includes a buffer that stores one or more episodes of EGM signals received from sensing circuit 204 and/or episodes of motion signals sensed by motion sensor 212 in response to control circuit 206 determining that a Vsense signal is an oversensed event signal. Memory 210 may store one or more motion metrics determined from the motion signal and used to identify false Vsense signals. A history of motion metrics corresponding to false Vsense signals and/or corresponding to Vsense signals determined to be true Vsense signals may be stored in memory 210 for transmission via telemetry circuit 208 and/or for use by control circuit 206 in establishing or adjusting one or more thresholds, ranges or values included in oversensing criteria applied to the motion metrics.

Telemetry circuit 208 includes a transceiver 209 and antenna 211 for transferring and receiving data via a radio frequency (RF) communication link. Telemetry circuit 208 may be capable of bi-directional communication with external device 20 (FIG. 1) as described above. Motion signals and cardiac electrical signals, and/or data derived therefrom such as sensed event data and motion metrics determined from the motion signal may be transmitted by telemetry circuit 208 to external device 20. Programmable control parameters and algorithms for sensing cardiac event signals and for identifying oversensed event signals as well as for controlling pacing therapies delivered by pulse generator 202 may be received by telemetry circuit 208 and stored in memory 210 for access by control circuit 206. In other examples, a communication circuit may be provided in pacemaker 14 for communication with atrial pacemaker 12 or other medical devices via TCC. TCC communication circuitry may include circuitry of pulse generator 202 for transmitting TCC signals and/or circuitry of sensing circuit 204 for receiving TCC signals transmitted to pacemaker 14.

Power source 214 provides power to each of the other circuits and components of pacemaker 14 as required. Power source 214 may correspond to battery subassembly 160 shown in FIG. 2 and may include one or more energy storage devices, such as one or more rechargeable or non-rechargeable batteries. The connections between power source 214 and other pacemaker circuits and components are not shown in FIG. 5 for the sake of clarity but are to be understood from the general block diagram of FIG. 5. Power source 214 may provide power as needed pulse generator 202, sensing circuit 204, telemetry circuit 208, memory 210 and motion sensor 212.

The functions attributed to ventricular pacemaker 14 herein may be embodied as one or more processors, controllers, hardware, firmware, software, or any combination thereof. Depiction of different features as specific circuitry is intended to highlight different functional aspects and does not necessarily imply that such functions must be realized by separate hardware, firmware or software components or by any particular circuit architecture. Rather, functionality associated with one or more circuits described herein may be performed by separate hardware, firmware or software components, or integrated within common hardware, firmware or software components. For example, a process of determining an oversensed event signal may be implemented in control circuit 206 executing instructions stored in memory 210 and relying on input from sensing circuit 204 and motion sensor 212. Providing software, hardware, and/or firmware to accomplish the described functionality in the context of any modern pacemaker, given the disclosure herein, is within the abilities of one of skill in the art.

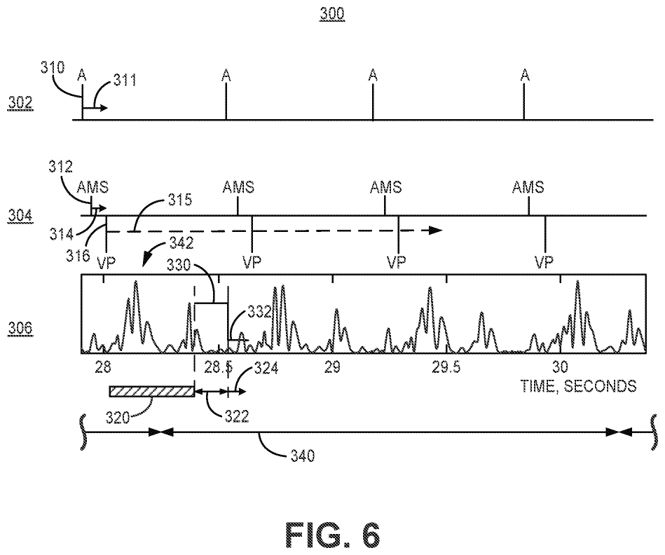

FIG. 6 is a timing diagram 300 of cardiac events and a motion signal 306 sensed over several cardiac cycles. Diagram 300 includes a marker channel 302 indicating the timing of atrial events as atrial event markers 310. The atrial event markers 310 may correspond to atrial pacing pulses. Atrial pacing pulses may be delivered by an atrial pacemaker, e.g., atrial pacemaker 12 shown in FIG. 1, that is co-implanted with ventricular pacemaker 14. In some examples, the ventricular pacemaker 14 may be configured to deliver atrial pacing pulses when implanted in the right atrium as described in conjunction with FIG. 3 above. In this case, control circuit 206 may start an AV pacing interval 311 in response to pulse generator 202 delivering an atrial pacing pulse during an atrial synchronous ventricular pacing mode.

In other examples, the atrial event markers 310 may indicate the timing of intrinsic P-waves, which may or may not be sensed by ventricular pacemaker 14. In some examples, atrial P-waves may be sensed by an atrial sensing channel of ventricular pacemaker sensing circuit 204, e.g., when ventricular pacemaker 14 is implanted in the right atrium. When control circuit 206 receives atrial sensed event signals from an atrial sensing channel of sensing circuit 204, control circuit 206 may start an AV pacing interval 311 for scheduling a ventricular pacing pulse 316 delivered by pulse generator 202 upon expiration of the AV pacing interval 311.

In other examples, when ventricular pacemaker 14 is implanted in the right ventricle, atrial event detector circuit 240 of control circuit 206 may sense atrial event signals from the motion signal 306. Marker channel 304 indicates the relative timing of atrial event signals sensed from motion signal 306 as atrial mechanical sense (AMS) markers 312. Marker channel 304 also includes ventricular pacing pulse (VP) makers 316 to indicate the timing of ventricular pacing pulses delivered by pulse generator 202. Control circuit 206 may start an AV pacing interval 314 in response to sensing an atrial event signal from the motion signal 306 for scheduling an atrial synchronous ventricular pacing pulse 316.

As shown in FIG. 6, the AV pacing interval 311 started by control circuit 206 in response to an atrial electrical event indicated by atrial event maker 310 may be different than the AV pacing interval 314 started in response to sensing the atrial mechanical event from motion signal 306. AV pacing interval 314 may be shorter than AV pacing interval 311 because the atrial mechanical event occurs slightly later (after atrial electrical depolarization) during the ventricular cycle. It is to be understood that pacemaker 14 may start one of AV pacing interval 311 in response to an atrial electrical event or AV pacing interval 314 in response to an atrial mechanical event and not necessarily both AV pacing interval 311 and AV pacing interval 314.

In response to delivering a ventricular pacing pulse 316 (e.g., at the expiration of AV pacing interval 311 or AV pacing interval 314), control circuit 206 may start a ventricular pacing interval 315. In the example shown, the ventricular pacing interval 315 is 1.5 seconds long and may be a LRI corresponding to a programmed lower rate (or base rate) of 40 beats per minute (bpm). The ventricular pacing interval 315 does not expire due to a subsequent ventricular pacing pulse being delivered in response to the next atrial sensed event signal during the ventricular pacing interval 315. While not illustrated in FIG. 6 for the sake of clarity, it is to be understood that ventricular pacing interval 315 can be restarted by control circuit 206 in response to each ventricular pacing pulse 316. Furthermore, it is to be understood that the ventricular pacing interval 315 may be adjusted to a rate smoothing interval based on the actual ventricular rate, e.g., based on a median ventricular cycle length determined between consecutive atrial synchronous ventricular pacing pulses. Control circuit 206 may be configured to determine a rate smoothing interval based on the actual ventricular rate to avoid abrupt changes in ventricular rate. The rate smoothing interval may be gradually adjusted toward the LRI corresponding to the programmed ventricular lower rate in the absence of sensed cardiac events. Example techniques for determining a rate smoothing interval and controlling ventricular pacing according to a rate smoothing interval are generally disclosed in U.S. Publication No. 2019/0321634, granted as U.S. Pat. No. 11,617,889 (Sheldon, et al.), incorporated herein by reference in its entirety.

When ventricular pacemaker 14 is configured to sense atrial event signals (from the motion signal 306 or from a sensed atrial electrical signal) for controlling atrial synchronous ventricular pacing pulses, atrial event detector circuit 240 (or sensing circuit 204) may start a post-ventricular atrial blanking period (PVAB) 320 in response to a delivered ventricular pacing pulse. The PVAB 320 may also be applied following a Vsense signal received from sensing circuit 204. Following a ventricular electrical event, e.g., pacing pulse 316 or a true Vsense signal, relatively large acceleration signals 342 are observed during the PVAB 320 due to ventricular contraction during the ventricular systolic phase of the cardiac cycle following the pacing-evoked or intrinsic ventricular electrical depolarization.

Upon expiration of the PVAB 320, atrial event detector circuit 240 may apply a first, high atrial event sensing threshold 330 to motion signal 306 during a passive ventricular filling phase window 322. As described in the above-incorporated U.S. Pat. No. 10,449,366 (Splett, et al.), when the atrial rate increases, the active filling phase of ventricular diastole associated with atrial mechanical contraction can coincide with the passive ventricular filling phase of ventricular diastole. When the active and passive filling phases occur concurrently, a relatively large acceleration signal can occur during the passive ventricular filling phase window 322, sometimes referred to as the “A3 window” because an “A3 signal” in the motion signal that corresponds to the passive filling phase of the ventricular cycle becomes fused with the A4 signal corresponding to atrial kick. If the motion signal 306 crosses the high atrial event sensing threshold 330 during the passive ventricular filling phase window 322, atrial event detector circuit 240 may sense an atrial event signal and trigger a ventricular pacing pulse by starting the AV pacing interval 314.

If the motion signal 306 does not cross the high atrial event sensing threshold 330 prior to the expiration of the passive filling phase window 322, atrial event detector circuit 240 may apply a second, low atrial sensing threshold amplitude 332 to the motion signal during an A4 sensing window 324. The A4 signal in the motion signal 306 is an acceleration signal corresponding to the atrial contraction or “atrial kick” that causes the active filling phase of ventricular diastole.

Following each ventricular pacing pulse 316 and following any true intrinsic R-waves (not shown in FIG. 6), relatively large acceleration signals 342 corresponding to ventricular mechanical systole may be observed in the motion signal 306. The acceleration signal peaks following a ventricular pacing pulse 316 may include an acceleration signal corresponding to ventricular contraction, sometimes referred to as an “A1 signal.” An acceleration signal that occurs with closure of the aortic and pulmonic valves, marking the approximate offset or end of ventricular mechanical systole, may follow the A1 signal and is sometimes referred to as the “A2 signal.” The PVAB 320 may be applied by atrial event detector circuit 240 to avoid sensing the large acceleration signals 342 associated with ventricular mechanical systole as an atrial event signal.

Control circuit 206 may determine a motion metric from the motion signal 306 sensed over a motion metric time interval 340 for use in detecting an oversensed event. The motion metric time interval 340 includes the motion signal sample points occurring during any PVABs that may be applied during the time interval 340. Control circuit 206 may determine the motion metric by determining an integration of the motion signal, also referred to herein as a summation metric, which may be determined by summing all or a portion of the rectified sample point amplitudes of the motion signal 306 received during motion metric time interval 340. In other examples, control circuit 206 may determine a motion metric by counting the number of sample points during the time interval 340 that have an amplitude that is greater than a threshold amplitude. In still other examples. control circuit 206 may determine the motion metric by summing the amplitudes of all sample points during the time interval 340 that have an amplitude that is greater than a threshold amplitude. The threshold amplitude may be applied to the motion signal sample points to filter out low amplitude event signals that do not correspond to a true ventricular systolic event signal. Sample points less than the threshold amplitude may be ignored for the purposes of determining a motion metric. In other examples, the motion metric may be determined as a ratio of a count of sample points greater than the threshold amplitude to a count of sample points less than the threshold amplitude.

The threshold amplitude may be set to be greater than a baseline noise amplitude, e.g., 0.1 to 2.0 m/s². In other examples the threshold amplitude may be set to be greater than an expected amplitude of A4 event signals. Control circuit 206 may be configured to establish the threshold amplitude based on an average, median or maximum atrial event signal peak amplitude determined from a specified number of most recent sensed A4 signals, e.g., greater than 1.0 to 3.0 m/s². In still other examples, the threshold amplitude may be set to be equal to the low atrial event sensing threshold amplitude 332 e.g., equal to 1.0 to 2.0 m/s².

The motion metric time interval 340 may be set to a motion metric time interval, e.g., 0.25 to 4 seconds or between 0.5 and 2 seconds. In other examples, the time interval 340 may set equal to the ventricular pacing interval 315, to the LRI, to a multiple of the LRI or the current ventricular pacing interval 315, e.g., to a multiple of a rate smoothing interval determined by control circuit 206. The motion metric time interval may be set to 0.5 to 3 times the ventricular pacing interval 315, as examples.

In some examples, the motion metric time interval 340 may be started by control circuit 206 independent of the timing of cardiac events or a starting time of a cardiac cycle, e.g., independent of the timing of atrial electrical sensed event signals or ventricular sensed event signals that may be received from sensing circuit 204, atrial mechanical sensed event signals produced by atrial event detector circuit 240, and/or ventricular pacing pulses delivered by pulse generator 202. A new motion metric time interval 340 may be started upon expiration of the immediately preceding motion metric time interval as illustrated in FIG. 6 to provide successive motion metric time intervals over which the motion signal is sensed and used to determine a motion metric for identifying oversensed events when a Vsense signal (not shown in FIG. 6) is received during the motion metric time interval 340.

The motion metric time interval 340 may be the same time interval used to determine a patient physical activity metric from which an SIR is determined in some examples. The motion metric and the patient physical activity metric may be the same metric in some examples. In other examples, the motion metric and the patient physical activity metric may be determined at the expiration of a common time interval 340 using a motion signal sensed during the time interval 340 but may be determined using a different axis signal or combination of axis signals received from motion sensor 212 and/or determined using a different method. For example, the motion metric may be determined from a motion signal filtered using different filtering properties than the motion signal used for determining the patient physical activity metric. In other examples, the motion metric may be determined using different operations, e.g., according to any of the examples described above involving counting, summing and/or applying a threshold amplitude to the motion signal sample points, than the method used for determining the patient physical activity metric.

In other examples, the motion metric time interval 340 may be started in response to a cardiac event, atrial or ventricular (sensed or paced). In some examples, motion metric time interval 340 can be started in response to a delivered ventricular pacing pulse and may be restarted in response to another ventricular pacing pulse that occurs before time interval 340 expires. If the time interval 340 does not expire before delivery of the next ventricular pacing pulse, the motion metric may not be determined for the partial motion metric time interval. In other examples, motion metric time interval 340 is started in response to a Vsense signal received from sensing circuit 204. In still other examples, the motion metric time interval 340 may be started in response to a Vsense signal sensed at a relatively short time interval from a most recent atrial event, e.g., atrial pacing pulse, sensed P-wave or sensed A4 signal. The relatively short time interval may be defined as a time interval that is 10 ms to 100 ms, as examples, extending from the atrial event. In another example, the motion metric time interval 340 may be started in response to more than one Vsense signal being received from the sensing circuit during a single atrial cycle (extending between two consecutive atrial events, which can be atrial pacing pulses and/or sensed P-waves and/or sensed A4 signals). The motion metric determined from the motion signal sensed during the motion metric time interval 340 may be used by control circuit 206 to determine that a Vsense signal is an oversensed event, e.g., a P-wave or atrial pacing pulse that is a cross-chamber oversensed event or another non-cardiac noise signal.

In the example shown in FIG. 6, ventricular pacing pulses 316 are delivered in response to each atrial sensed event (either the A4 signals sensed from motion signal 306 or the atrial electrical events shown by marker channel 302) with no ventricular sensed event signals received by control circuit 206. As such, no oversensed events are determined. When a Vsense signal does not occur during a motion metric time interval 340, the motion metric may not be determined by control circuit 206 for that motion metric time interval, unless otherwise needed for other pacemaker functions, e.g., for determining an SIR and a rate response pacing interval.

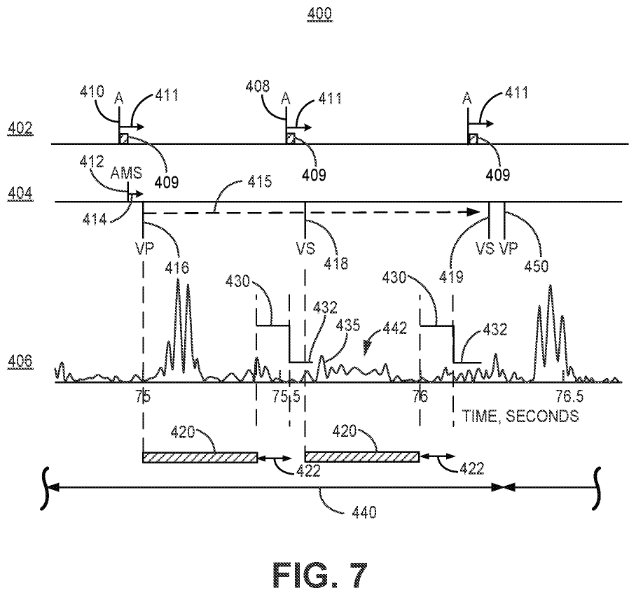

FIG. 7 is a timing diagram 400 of cardiac events and a motion signal 406 occurring over several cardiac cycles according to another example. An atrial marker channel 402 depicts the atrial event markers 410, which may correspond to a delivered atrial pacing pulse or an intrinsic atrial P-wave. Marker channel 404 depicts the timing of sensed A4 signals (AMS) 412, delivered ventricular pacing pulses 416 and 450, and Vsense signals 418 and 419 received from sensing circuit 204. As described above in conjunction with FIG. 6, when ventricular pacemaker 14 is implanted in the right atrium, atrial pacing pulses may be delivered by pulse generator 202 and/or intrinsic atrial P-waves may be sensed by the atrial channel of sensing circuit 204. Pulse generator 202 may deliver a ventricular pacing pulse 416 after an AV pacing interval 411 started by control circuit 206 in response to the atrial electrical event denoted by atrial event markers 410.

When an atrial pacemaker 12 is included in IMD system 10, with ventricular pacemaker 14 implanted in the right ventricle, the atrial marker channel 402 may depict atrial electrical events, sensed by the atrial pacemaker or corresponding to delivered atrial pacing pulses. In this case, the ventricular pacemaker 14 may sense atrial mechanical events, as denoted by atrial mechanical sense markers 412 from motion signal 406 for starting an AV pacing interval 414 to schedule atrial synchronous ventricular pacing pulses 416. In other examples, ventricular pacemaker 14 may receive a communication signal from atrial pacemaker 12 (or another device sensing P-waves and/or delivering atrial pacing pulses) indicating the timing of atrial events to trigger ventricular pacemaker 14 to deliver an atrial synchronous ventricular pacing pulse.

In any of these examples of ventricular pacemaker 14 being implanted in the right atrium or in the right ventricle, sensing circuit 204 may oversense atrial electrical events as false R-waves. For example, an atrial pacing pulse delivered by atrial pacemaker 12 may be oversensed as a false R-wave by sensing circuit 204 of ventricular pacemaker 14. When ventricular pacemaker 14 is implanted in the right atrium, a near field P-wave may be oversensed as a false R-wave by the ventricular sensing channel of sensing circuit 204. Such CCOS events may cause control circuit 206 of ventricular pacemaker 14 to withhold or inhibit a scheduled ventricular pacing pulse when a ventricular pacing pulse may actually be needed in order to prevent asystole.

For example, as shown in FIG. 7, a Vsense signal 418 is received by control circuit 206 from sensing circuit 204 near the time of the atrial electrical event 408. The Vsense signal 418 is a CCOS event. The Vsense signal 418 may prevent control circuit 206 from starting an AV interval 411 for scheduling an atrial synchronous ventricular pacing pulse or cancel a scheduled atrial synchronous ventricular pacing pulse if the Vsense signal 418 is received during the AV interval 411. When ventricular pacemaker 14 relies on atrial mechanical event sensing from the motion signal 406, a Vsense signal 418 received during the AV interval 414 may cause control circuit 206 to withhold a ventricular pacing pulse scheduled at the expiration of the AV interval 414. In the example shown, the A4 signal 435 that follows atrial electrical event 408 may not be sensed by atrial event detector circuit 240. Control circuit 206 may start a PVAB 420 in response to the Vsense signal 418. The A4 signal 435 may occur during the PVAB 420 started in response to Vsense signal 418 such that it is not sensed and a ventricular pacing pulse is not scheduled at an AV pacing interval by control circuit 206 due to the false Vsense signal 418. When the Vsense signal 418 is not identified as an oversensed event, control circuit 206 would normally restart the ventricular pacing interval 415 in response to receiving the Vsense signal 418. As a result, a ventricular pacing pulse may be inhibited or withheld due to the false Vsense signal 418. If the atrial electrical event 408 is not conducted to the ventricles, the motion signal amplitude remains low where the A1 and A2 acceleration signals would normally occur as indicated by arrow 442.

In the example shown, at the end of the PVAB 420 started in response to the false Vsense signal 418, atrial event detector circuit 240 may apply the high atrial event sensing threshold 430 and low atrial event sensing threshold 432 to the motion signal 406 as described above in conjunction with FIG. 6. However, because the false Vsense signal started the PVAB 420 that is followed by the subsequent passive ventricular filling phase window 422, an A4 signal does not occur during the passive ventricular filling phase window 422. The A4 signal occurs after the next atrial electrical event, which in this example is also a CCOS event as shown by the ventricular sensed event signal 419, which prevents sensing of the next A4 signal as well. In this way, a CCOS event may result in undersensing of atrial event signals, e.g., A4 signals, on one or more subsequent cardiac cycles due to the application of atrial event sensing thresholds 430 and 432 at times during the cardiac cycle that are mis-timed with respect to the atrial rhythm on subsequent cardiac cycles.

According to the techniques disclosed herein, however, control circuit 206 may identify the Vsense signal 418 as an oversensed event by determining the motion metric at the expiration of the motion metric time interval 440. In response to the Vsense signal 418 being received during the motion metric time interval 440, control circuit 206 may compare the motion metric determined from the motion signal sensed during all or at least a portion of time interval 440 to oversensing criteria. When the motion metric is less than an oversensing threshold, for example, due to the low amplitude motion signal 442 in the absence of a true ventricular event, control circuit 206 may detect the Vsense signal 418 as a CCOS event. The motion metric determined from the motion signal sensed over time interval 440 that includes a false Vsense signal may be less than a motion metric determined from the same time interval 440 when a true ventricular event occurs during the motion metric time interval 440.

In response to detecting the Vsense signal 418 as being an oversensed event, control circuit 206 may perform a ventricular pacing response and/or a ventricular sensing response. In some examples, control circuit 206 does not restart the running ventricular pacing interval 415 in response to the Vsense signal 418 until the Vsense signal 418 is verified to be a true Vsense signal based on the motion metric determined at the end of the motion metric time interval 440 not meeting oversensing criteria. Pulse generator 202 may deliver a ventricular pacing pulse 450 upon expiration of the ventricular pacing interval 415 or upon expiration of the motion metric time interval 440 (as shown). In some examples, control circuit 206 may enable safety pacing in response to detecting an oversensed event. In some examples, ventricular pacing pulse 450 may be delivered as a safety pacing pulse within the absolute physiological refractory period of the next Vsense signal 419. In this way, if the Vsense signal 419 is also an oversensed event, asystole is avoided. The ventricular pacing pulse 450 is synchronized to the atrial electrical event that is oversensed as a false R-wave by being delivered at a safety pacing interval from the Vsense signal, which is actually an atrial electrical event. In this way, AV synchrony can be restored to promote regaining regular sensing of the atrial event signals, electrical or mechanical, during subsequent ventricular cycles.

In other examples, control circuit 206 may switch to an asynchronous pacing mode to deliver ventricular pacing pulses at a regular rate for a predetermined number of pacing pulses or predetermined time interval. In another example, control circuit 206 may switch to a ventricular triggered pacing mode to provide ventricular pacing pulses within a safety pacing interval of a Vsense signal to avoid asystole in case a Vsense signal is an oversensed event. Control circuit 206 may switch back to the atrial synchronous ventricular pacing mode after the predetermined time interval or a fixed number of asynchronous ventricular pacing pulses or after no false Vsense signals are identified for at least a motion metric time interval or specified number of pacing cycles.

As further described below, control circuit 206 may adjust a ventricular sensing control parameter, such as the ventricular sensitivity and/or a post-atrial ventricular blanking period 409, to reduce the likelihood of oversensing subsequent atrial electrical events as false R-waves. Ventricular pacemaker 14 can be configured to identify atrial events. Atrial events may be identified by sensing atrial P-waves by sensing circuit 204. Atrial events may be identified by sensing A4 signals from the motion signal shown by AMS markers 412. Atrial events may be identified from a communication signal received by ventricular pacemaker 14 from another medical device indicating the timing of the atrial events. Atrial events may be identified as an atrial pacing pulse delivered by pulse generator 202. Control circuit 206 may start a post-atrial ventricular blanking period 409 in response to identifying an atrial event. A ventricular event may not be sensed by sensing circuit 204 during the post-atrial ventricular blanking period. When a Vsense signal is identified as an oversensed event, a ventricular sensing response may include increasing the post-atrial ventricular blanking period to reduce the likelihood of oversensing the atrial event as a false R-wave.

In still other examples, control circuit 206 may provide a ventricular sensing response by enabling dual sensor sensing of ventricular event signals. After enabling dual sensor sensing of ventricular event signals, a ventricular event may be sensed or responded to by control circuit 206 only when both the cardiac electrical signal and the motion signal 406 cross respective ventricular event sensing thresholds within a threshold time of each other, for example.

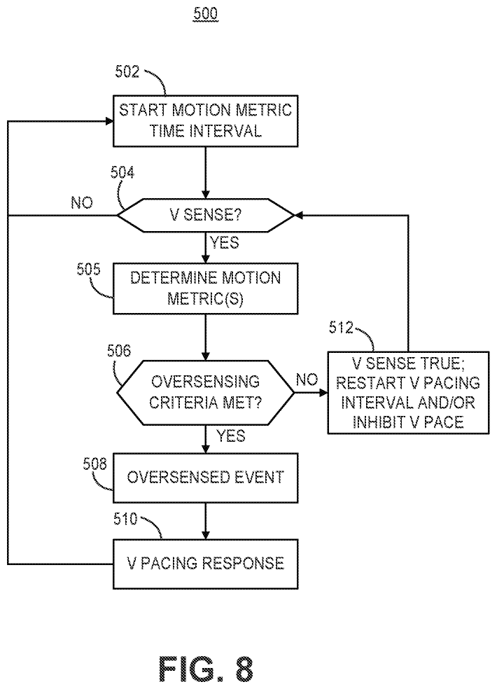

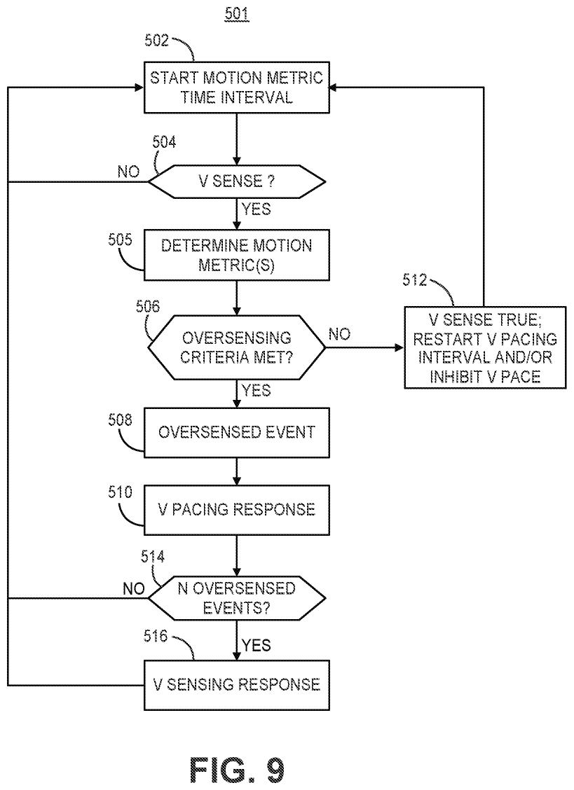

FIG. 8 is a flow chart 500 of a method for detecting oversensed events by processing circuitry of ventricular pacemaker 14 according to some examples. At block 502, control circuit 206 starts a specified motion metric time interval. The motion signal sensed during the motion metric time interval may be buffered in memory 210. In other examples, motion signal sample points may be used by control circuit 206 as the motion signal is received from motion sensor 212 over the motion metric time interval for determining the motion metric. The motion metric time interval may be set to 0.3, 0.5, 1, 1.5, 2.0, 2.5 or 3 seconds as examples. The motion metric time interval may be set to a multiple of the ventricular pacing interval, e.g., 0.25, 0.5, 1, 1.5, or 2 times the ventricular pacing interval currently in effect in other examples. The ventricular pacing interval currently in effect may be, for example, the programmed LRI, a rate smoothing interval or a rate response interval.

The motion metric time interval may be started at block 502 upon expiration of a preceding motion metric time interval, e.g., as illustrated by time interval 340 in FIG. 6 and time interval 440 in FIG. 7. In this case, the motion metric time interval may start and end independent of the relative timing of any cardiac events or cardiac cycles. In some examples, the motion metric time interval may extend over multiple cardiac cycles.

In other examples, the motion metric time interval is started at block 502 in response to the sensing circuit 204 sensing a ventricular event signal, e.g., when control circuit 206 receives a Vsense signal from sensing circuit 204. In still other examples, the motion metric time interval may be started in response to a ventricular pacing pulse delivered by pulse generator 204. If another ventricular pacing pulse is delivered before the motion metric time interval expires, the motion metric time interval may be restarted in response to the ventricular pacing pulse without necessarily determining the motion metric.

In still other examples, the motion metric time interval may be started at a predetermined delay after a delivered ventricular pacing pulse. For example, the motion metric time interval may be started after a ventricular blanking period to exclude the ventricular event signal, e.g., the A1 and A2 signals, of the motion signal following the ventricular pacing pulse. In this way, the motion metric determined at the end of the motion metric time interval may be more sensitive to discriminating between a false Vsense signal and a true Vsense signal when a Vsense signal occurs during the motion metric time interval. In other examples, control circuit 206 may start the motion metric time interval in response to an atrial event, e.g., a delivered atrial pacing pulse, a sensed P-wave, a sensed A4 signal, or a received communication signal indicating the timing of an atrial event.

If a Vsense signal is not received during the motion metric time interval, as determined at block 504, the next motion metric time interval may be started (or restarted) at block 502 according to any of the examples given above. For example, the next motion metric time interval may be started immediately upon expiration of the preceding motion metric time interval, in response to the next delivered ventricular pacing pulse, in response to sensing circuit 204 sensing a ventricular event signal, or in response to an atrial event. The motion metric is not necessarily determined by control circuit 206 if sensing circuit 204 does not sense a ventricular event signal during the motion metric time interval. If the motion signal is buffered in memory 210 during the motion metric time interval, it may be discarded. When the motion metric is being determined as motion signal sample points are received by control circuit 206, the motion metric determined upon expiration of the motion metric time interval may be discarded. However, the buffered motion signal may be used and/or the motion metric may still be determined by control circuit 206 if control circuit 206 is using the motion metric for other purposes, e.g., for determining a patient physical activity metric and associated SIR controlling rate response pacing. In some examples, the motion metric may be determined and stored in memory 210 and can be used in updating an oversensing detection threshold in some examples if a Vsense signal was not received during the motion metric time interval.

If control circuit 206 determines at block 504 that a Vsense signal is received from sensing circuit 204 during the motion metric time interval (or if a Vsense signal caused control circuit 206 to start the motion metric time interval), control circuit 206 determines the motion metric from the motion signal received from motion sensor 212 at block 505. As indicated above, control circuit 206 may determine the motion metric as the motion signal sample points are being received from motion sensor 212. In this case, the motion metric is determined and immediately available upon expiration of the motion metric time interval. The motion metric may be determined by summing sample point amplitudes of all sample points of the rectified motion signal sensed during the motion metric time interval. The motion metric may be determined by summing all sample point amplitudes of the rectified motion signal that are greater than a threshold amplitude sensed during the motion metric time interval. In still other examples, the motion metric may be determined by counting sample points having an amplitude greater than a threshold amplitude that are sensed during the motion metric time interval.

In some examples, both a summation and a count of motion signal sample points may be determined at block 505 by control circuit 206. When a true ventricular event is sensed during the motion metric time interval, the motion metric determined as a summation of all sample points is expected to be greater than a threshold value. However. a relatively low baseline portion of the motion signal is expected during the cardiac cycle between cardiac events. As such, a count of motion signal sample points that are greater than a threshold amplitude may be determined to distinguish from a high value of the summation of sample point amplitudes that is caused by noise or non-cardiac motion. The count of motion signal sample points having an amplitude greater than a threshold amplitude may distinguish between sample points corresponding to true ventricular systolic motion signals (e.g., A1 and A2 signals) compared to other lower amplitude signals caused by other non-ventricular motion and baseline noise.

When at least one Vsense signal is received during the motion metric time interval, control circuit 206 may compare the motion metric(s) determined at block 505 to oversensing criteria at block 506. The oversensing criteria may include one or more thresholds, values or ranges that may be applied to a respective motion metric determined at block 505 to detect when the amount of motion present in the motion signal likely corresponds to ventricular systolic contraction, indicating that the Vsense received during the motion metric time interval is a true Vsense signal. For example, if the motion metric is determined as a summation metric by summing the amplitude of all sample points sensed during the motion metric time interval, and the motion metric is greater than an oversensing threshold, the Vsense signal may be determined to be a valid Vsense signal by control circuit 206 at block 512.

Depending on the time duration of the motion metric time interval, multiple ventricular events (true and/or oversensed) could occur during the motion metric time interval. For example, one or more ventricular pacing pulses and/or one or more Vsense signals could occur during a motion metric time interval. In this case, the motion metric may be normalized by the number of ventricular events occurring during the motion metric time interval or the motion metric may be compared to an oversensing threshold that is scaled according to the number of ventricular events during the motion metric time interval.

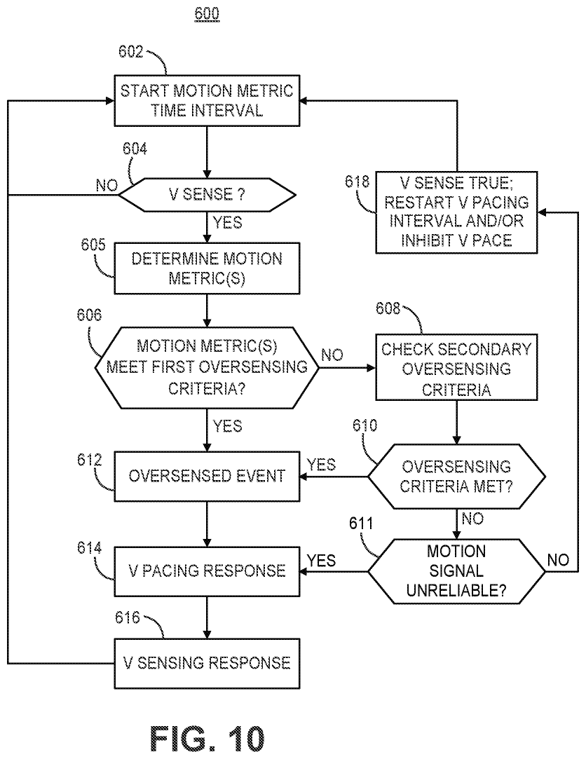

In some examples, when the motion metric determined as a summation metric is greater than the oversensing threshold, patient physical activity may be contributing to the motion signal amplitude during the motion metric time interval. In some examples, if physical activity of the patient is contributing to the motion metric being greater than the oversensing threshold it may be assumed that the patient is unlikely to be experiencing asystole such that the Vsense signal can be determined to be a true R-wave at block 512. In other examples, if the motion metric, e.g., determined as a summation of sample point amplitudes, is greater than an oversensing threshold, patient physical activity may be contributing to the motion metric determined from sample points spanning the entirety of the motion metric time interval. The Vsense signal could still be false even though the motion metric represents a relatively high level of motion during the motion metric time interval. As such, secondary oversensing criteria may be applied to the motion signal sensed during the motion metric time interval or a portion thereof for distinguishing between a motion metric representative of true ventricular motion associated with the Vsense signal and a motion metric that is greater than an oversensing threshold due to other non-cardiac motion signals present during the motion metric time interval. Examples of secondary oversensing criteria are described below in conjunction with FIG. 10.

Additionally or alternatively, a motion metric may be determined at block 505 as a sample point count of all motion signal sample points (of the rectified motion signal) that are greater than a threshold amplitude. Control circuit 206 may determine if the sample point count is greater than a threshold count value representative of ventricular systolic motion, e.g., as represented by A1 and A2 signals, present in the motion signal during the motion metric time interval. When the sample point count is greater than the threshold count value and/or the summation metric is greater than an oversensing threshold amplitude, control circuit 206 may determine that the Vsense signal received during the motion metric time interval is a true R-wave at block 512.

In response to determining that the Vsense signal is true, control circuit 206 may restart a ventricular pacing interval at block 512 to inhibit ventricular pacing. A ventricular pacing interval, which may be an LRI, a temporary rate response interval or rate smoothing interval, may be restarted to have an effective starting time at the time of the Vsense signal in some examples. In other examples, the ventricular pacing interval may be restarted at the expiration of the motion metric time interval following the Vsense signal even though restarting the ventricular pacing interval is delayed by the time taken to determine that the Vsense signal is valid.

In some examples, if a previously started ventricular pacing interval expires after the Vsense signal is received by control circuit 206 but before the motion metric time interval expires, control circuit 206 may withhold the pending, scheduled ventricular pacing pulse. At block 512, the pending ventricular pacing pulse can be inhibited (canceled) by control circuit 206 due to the Vsense signal being determined to be a valid sensed ventricular event. The pending pacing pulse may be cancelled and the ventricular pacing interval may be restarted. Control circuit 206 may return to block 502 to start the next motion metric time interval according to any of the examples described above.

When the oversensing criteria are met at block 506, control circuit 206 may determine that the ventricular event sensed by sensing circuit 204 is an oversensed event at block 508, which may be an oversensed atrial pacing pulse or an oversensed P-wave or a non-cardiac noise signal. Control circuit 206 may log the oversensed event detection in memory 210. Control circuit 206 may count the number of oversensed event detections, e.g., since the last interrogation session and/or the number of consecutively identified oversensed events. Control circuit 206 may store in memory 210 a time segment of the cardiac electrical signal sensed by sensing circuit 204 from which the oversensed event was sensed. The cardiac electrical signal(s) and/or oversensed event detection history logged in memory 210 may be transmitted to external device 20 (FIG. 1) for review by a clinician or other user for use in selecting ventricular event sensing control parameters, such as a programmed sensitivity.

In response to determining the oversensed event at block 508, control circuit 206 may provide a ventricular pacing response at block 510. Control circuit 206 may control pulse generator 202 to deliver at least one ventricular pacing pulse at block 510. In some examples, a ventricular pacing interval, which may be an LRI, rate smoothing interval or rate response interval as examples, that is running at the time of the Vsense signal determined to be an oversensed event may be allowed to continue running without being restarted. Pulse generator 202 may deliver the ventricular pacing pulse scheduled at the expiration of the ventricular pacing interval at block 510.

In other examples, pulse generator 202 may deliver a ventricular pacing pulse upon expiration of the motion metric time interval and determination of the oversensed event. If a ventricular pacing interval expired after the Vsense signal during the motion metric time interval such that a pending pacing pulse is waiting to be delivered by pulse generator 202, pulse generator 202 may deliver the pending pacing pulse upon determination of the oversensed event. In still other examples, control circuit 206 may control pulse generator 202 to start a safety pacing interval in response to the oversensed event determination. The safety pacing interval may be set to 500 ms, 750 ms, 800 ms, 1000 ms or any other time interval from the expiration of the motion metric time interval. If the Vsense signal is received near the expiration of the motion metric time interval, control circuit 206 may schedule a ventricular pacing pulse after a safety pacing interval to avoid delivering a ventricular pacing pulse during the repolarization phase of the ventricular cycle in case the Vsense signal is true and falsely determined to be an oversensed event. When a ventricular pacing pulse is delivered during the vulnerable phase that corresponds to the first part of the T-wave in the cardiac electrical signal and the pacing pulse is of sufficient magnitude to cause ventricular capture during this relative refractory state, ventricular fibrillation could be induced in some patients.

The safety pacing interval could be started by control circuit 206 in response to the Vsense signal received during the motion metric time interval. In other examples, the safety pacing interval can be started upon determining that the Vsense signal is an oversensed event. In this way, in some examples control circuit 206 may schedule a ventricular pacing pulse to occur no earlier than a safety pacing interval after the Vsense signal when it is determined to be an oversensed event upon expiration of the motion metric time interval. In some examples, the motion metric time interval may be set equal to a safety pacing interval.

Pulse generator 202 may deliver the pending or scheduled ventricular pacing pulse at block 510 according to an oversensed event pacing response. If an atrial event signal, e.g., an A4 signal or a P-wave, is sensed before the scheduled or pending ventricular pacing pulse is delivered, a triggered atrial synchronous ventricular pacing pulse may be delivered at an AV interval from the sensed atrial event signal. In this case, any ventricular pacing pulse that is scheduled or pending according to the oversensed event pacing response may be cancelled. A ventricular pacing interval may be restarted in response to delivery of the atrial synchronous ventricular pacing pulse.

In some examples, control circuit 206 may switch to a temporary asynchronous ventricular pacing mode at block 510. For example, control circuit 206 may switch from a VDD, VDI, or VVI pacing mode that includes ventricular event sensing to a pacing mode that does not include ventricular event sensing, such as a VOO pacing mode. Control circuit 206 may operate in the asynchronous ventricular pacing mode without ventricular event sensing to enable ventricular pacing pulse delivery by pulse generator 202 for multiple ventricular cycles, e.g., at the LRI and/or rate smoothing interval(s), to promote a regular ventricular rate and avoid symptomatic asystole. After a predetermined number of ventricular paced cycles or specified time interval following the oversensed event, control circuit 206 may switch back to a ventricular pacing mode that includes ventricular event signal sensing, e.g., an atrial synchronous ventricular pacing mode (e.g., a VDD pacing mode) or an asynchronous ventricular pacing mode with ventricular event sensing, e.g., a VVI pacing mode. After delivering one or more ventricular pacing pulses in accordance with an oversensing ventricular pacing response, control circuit 206 may return to block 502 to start the next motion metric time interval as described above.

FIG. 9 is a flow chart 501 of a method that may be performed by pacemaker 14 for detecting and responding to oversensing according to another example. Identically numbered blocks in FIG. 9 correspond to like-numbered blocks shown in FIG. 8 and are described above. In some examples, in addition to or alternatively to performing a ventricular pacing response (block 510) in response to detecting an oversensed event, control circuit 206 may perform a ventricular sensing response at block 516. Control circuit 206 may determine if a threshold number of oversensed events are detected at block 514. The threshold number of oversensed events may be required to be detected consecutively, e.g., two, three, four or more consecutive oversensed events. In other examples, the threshold number of oversensed events may be required to be a threshold percentage of most recent ventricular events (including both ventricular pacing pulses and Vsense signals), e.g., 10%, 20%, 25% or 30% or a higher percentage of the most recent five, eight, twelve or other specified number of ventricular events. In another example, the threshold number of oversensed events may be required to be a threshold percentage of most recent Vsense signals, e.g., at least 30%, 40%, 50%, or any other selected percentage of the most recent four, five, eight, twelve or other specified number of Vsense signals. In yet another example, the threshold number of oversensed events may be a threshold number of oversensed events within a specified time interval, e.g., 2, 3, 4, 5, 10, 20, 100 or other specified number of oversensed events within 10 seconds, 30 seconds, 60 seconds, 120 seconds, one hour, 24 hours, or other specified time interval. The foregoing examples are illustrative in nature and not intended to be limiting.

When an oversensed event is identified, or when a threshold number of oversensed events are identified as determined at block 514, control circuit 206 may perform a ventricular sensing response at block 516 by adjusting one or more ventricular sensing control parameters. In some examples, a ventricular sensitivity setting is increased. The ventricular sensitivity setting is set to a voltage amplitude, e.g., 0.45 to 11.3 millivolts, and is the lowest amplitude of the cardiac electrical signal that can be sensed by sensing circuit 204 as a ventricular event signal. When oversensing occurs, the sensitivity setting may be increased so that sensing circuit 204 is less sensitive to sensing low amplitude signals as ventricular event signals.

The sensitivity setting may be increased, e.g., by a predetermined increment or to the next higher sensitivity setting available. In some examples, control circuit 206 may track a mean, median or minimum peak amplitude of sensed R-waves. For example, sensing circuit 204 may include a peak track and hold circuit for determining a peak amplitude of the rectified cardiac electrical signal sensed from a ventricular sensing electrode vector following an R-wave sensing threshold crossing (that causes event detector 224 to generate a Vsense signal). The peak amplitude may be used by sensing circuit 204 to set a starting amplitude of the R-wave sensing threshold applied to the cardiac electrical signal after the Vsense signal, e.g., following a ventricular blanking period.

Sensing circuit 204 may automatically adjust the R-wave sensing threshold amplitude applied to the cardiac electrical signal during the ventricular cycle following a Vsense signal until the next R-wave sensing threshold crossing occurs or until the next ventricular pacing pulse, whichever is first. Sensing circuit 204 may decrease the R-wave sensing threshold amplitude from the starting amplitude to the minimum R-wave sensing threshold amplitude, e.g., equal to the sensitivity setting. In some examples, the R-wave sensing threshold amplitude is decreased from the starting amplitude to the sensitivity setting according to one or more decay rates until a next R-wave sensing threshold crossing occurs or a ventricular pacing pulse is delivered, whichever is first.

Control circuit 206 may determine a mean, median, minimum or other representative R-wave peak amplitude from the peak amplitudes determined by sensing circuit 204 and used in setting the starting R-wave sensing threshold amplitude. For example, control circuit 206 may determine a representative peak amplitude from the most recent 5 to 20 peak amplitudes or other specified number of the most recent peak amplitudes following Vsense signals generated by sensing circuit 204 that are determined not to be oversensed events. At block 516, control circuit 206 may increase the ventricular sensitivity setting to a percentage of or to a specified amount less than the representative R-wave peak amplitude in response to detecting at least one oversensed event. In other examples, control circuit 206 may determine the maximum peak amplitude of the cardiac electrical signal following one or more Vsense signals that are identified as oversensed events and set the sensitivity setting to be greater than the maximum peak amplitudes of the oversensed events (up to a maximum sensitivity limit).

After adjusting the sensitivity setting, control circuit 206 may return to block 502. If one or more additional oversensed events are detected after increasing the sensitivity setting, control circuit 206 may switch to an asynchronous ventricular pacing mode without ventricular sensing, e.g., a VOO pacing mode, for a predetermined number of ventricular pacing pulses or a predetermined time interval or provide another ventricular pacing response to oversensed events according to any of the examples presented herein. In some examples, control circuit 206 may increase the sensitivity setting a maximum number of times or up to a maximum sensitivity setting at block 516.

In addition to or alternatively to adjusting the sensitivity setting at block 516, control circuit 206 may switch to dual sensor ventricular event sensing. For example, control circuit 206 may switch to sensing ventricular event signals using both the motion signal and the cardiac electrical signal. When an R-wave sensing threshold crossing occurs, control circuit 206 may verify that the motion signal crosses a ventricular event sensing threshold amplitude within a post-sense ventricular time window of the R-wave sensing threshold crossing. The ventricular event sensing threshold amplitude applied to the motion signal may be set to an amplitude that enables sensing of relatively large amplitude A1 and/or A2 signals that correspond to ventricular systole, for example. Control circuit 206 may determine that a true R-wave is sensed when both of the cardiac electrical signal and the motion signal meet a ventricular event sensing threshold amplitude, match a ventricular event morphology, meet a ventricular event maximum slope or other feature or combination of features during dual sensor ventricular event sensing. Dual sensor ventricular event sensing may be enabled indefinitely (e.g., until programmed off by a user), for a predetermined time interval, or until all (or at least a majority of) Vsense signals produced by sensing circuit 204 are determined to be true R-waves based on an analysis of the motion signal during a post-sense ventricular time window and/or based on motion metrics.

In another example, the ventricular sensing response performed by control circuit 206 at block 516 may include increasing a post-atrial ventricular blanking period. When a P-wave is sensed (e.g., by sensing circuit 204), an atrial pacing pulse is delivered (e.g., by pulse generator 202), or an A4 signal is sensed from the motion signal, e.g., by control circuit 206, sensing circuit 204 may apply a post-atrial ventricular blanking period. The post-atrial ventricular blanking period may be increased in response to identifying one or more oversensed events. The post-atrial ventricular blanking period may be 20 to 200 ms long and may be increased by 25 to 100 ms in various examples. An R-wave sensing threshold crossing during the post-atrial ventricular blanking period may be ignored (no Vsense signal generated) by sensing circuit 204, or a Vsense signal received by control circuit 206 from sensing circuit 204 during the post-atrial ventricular blanking period may be ignored by control circuit 206.

Control circuit 206 may increase the time that a ventricular sensing response is in effect each time an episode or threshold number of oversensed events is detected. For example, a higher sensitivity setting and/or dual sensor ventricular event sensing may initially be applied for 1 minute following a threshold number or frequency of oversensed event detections. The next time the threshold number or frequency of oversensed event detections is determined by control circuit 206, control circuit 206 may increase the time, e.g., double the time, that the increased ventricular sensitivity setting and/or dual sensor ventricular sensing is in effect. When a maximum ventricular sensing response time is reached, e.g., 16 minutes, 32 minutes, 128 minutes or other selected time, control circuit 206 may make the ventricular sensing response permanent or indefinite until reprogrammed by a user. In other examples, control circuit 206 may switch to an operating mode for controlling ventricular pacing pulses that does not rely of R-wave sensing.

In some examples, the ventricular sensitivity setting may be increased one or more times first by control circuit 206 in response to oversensed event detection, and, when a maximum ventricular sensitivity setting is reached and oversensed events are still being detected, control circuit 206 may enable dual sensor ventricular event sensing. Alternatively, the ventricular sensitivity setting may be increased by control circuit 206 in response to detecting oversensed events after dual chamber ventricular event sensing is enabled for a maximum number of times or a maximum time interval.

While both of a ventricular pacing response and a ventricular sensing response are shown in flow chart 501, it is to be understood that in various examples control circuit 206 may perform one of the ventricular pacing response or the ventricular sensing response, but not necessarily both, after determining that at least one Vsense signal is an oversensed event.

FIG. 10 is a flow chart 600 of a method for detecting and responding to oversensed events according to another example. At block 602, control circuit 206 may start a motion metric time interval according to any of the examples described above. When a Vsense signal is received (block 604) during the motion metric time interval, control circuit 206 may determine at least one motion metric (block 605) and compare the motion metric to oversensing criteria at block 606.

The motion metric(s) may be determined at block 605 by control circuit 206 as a summation metric and/or a sample point count as described above. When the summation metric and/or the sample point count is/are less than a respective threshold, as determined at block 606, control circuit 206 may determine that first oversensing criteria are met at block 606. The Vsense signal received from sensing circuit 204 during the motion metric time interval can be identified as an oversensed event at block 612. Control circuit 206 may perform a ventricular pacing response (block 614) and/or a ventricular sensing response (block 616) according to any of the examples described above in conjunction with FIGS. 8 and 9. The first oversensing criteria applied to the motion signal at block 606 by control circuit 206 may be applied by determining at least one motion metric based on sample points spanning the entirety of the motion metric time interval and comparing the at least one motion metric to an oversensing threshold in some examples.

When a motion metric determined at block 605 based on the motion signal spanning the entire motion metric time interval is greater than a respective oversensing threshold at block 606, control circuit 206 may apply secondary oversensing criteria at block 608. Non-cardiac motion may be contributing to the motion signal causing a motion metric to be greater than the oversensing threshold such that the first oversensing criteria are not met, but the Vsense signal received from sensing circuit 204 may be a falsely sensed R-wave. As such, control circuit 206 may be configured to perform further analysis of the motion signal for verifying that the received Vsense signal is a true R-wave when the first oversensing criteria are not met at block 606 (“no” branch of block 606). When secondary oversensing criteria are met as determined at block 608, control circuit 206 may still determine that the Vsense signal received from sensing circuit 204 is an oversensed event at block 612. A pacing and/or sensing response may be performed at respective blocks 614 and/or 616 as generally described above.

When the secondary oversensing criteria are not met (“no” branch of block 610), control circuit 206 can determine that the Vsense signal corresponds to a true R-wave at block 618. Control circuit 206 may restart a ventricular pacing interval in response to the verified Vsense signal and/or inhibit a scheduled or pending ventricular pacing pulse in response to the Vsense signal.

In some examples, if the secondary oversensing criteria are not met, control circuit 206 may determine if the motion signal is unreliable for confirming that a Vsense signal is a true R-wave. For example, if the first oversensing criteria are not met at block 606 and/or the secondary oversensing criteria are not met at block 610, control circuit 206 may compare the motion metric to a second higher threshold. When non-cardiac motion, e.g., due to patient activity, is contributing to the motion signal, the motion metric may be higher than a second higher threshold making ventricular mechanical event signals indiscriminate from non-cardiac motion present in the motion signal. In this case, control circuit 206 may determine that the motion signal is not reliable for confirming the Vsense signal. Control circuit 206 may provide a ventricular pacing response at block 614 by delivering a ventricular safety pacing pulse or switching to a triggered ventricular pacing mode (e.g., VVT) or a ventricular pacing mode without ventricular sensing (e.g., VOO pacing mode). If control circuit 206 determines that the motion signal is reliable at block 611, e.g., based on the motion metric being less than a second higher threshold, control circuit 206 may determine that the Vsense signal is true at block 618.

Analyzing the motion signal for assessing secondary oversensing criteria at block 608 may include analysis of the motion signal sample points spanning a portion of the motion metric time interval that is less than the entire motion metric time interval or a different portion of the motion metric time interval used to determine if the first oversensing criteria were met at block 606. For example, control circuit 206 may set a ventricular systole window following the Vsense signal received at block 604 in some examples. The motion signal sensed during the motion metric time interval may be buffered at least for a ventricular systole window in response to the Vsense signal. The ventricular systole window may extend, for example, 200 to 500 ms following the Vsense signal. Control circuit 206 may be configured to apply secondary oversensing criteria to the motion signal sensed during the ventricular systole window. For example, control circuit 206 may determine at least a second motion metric from the motion signal sensed during the ventricular systole window at block 608 for determining if oversensing criteria are met when a first motion metric does not meet the first oversensing criteria at block 606.

In some examples, control circuit 206 may determine a second motion metric as the maximum peak amplitude of the motion signal during the ventricular systole window. When the motion signal maximum amplitude is less than the ventricular systolic event threshold amplitude during the ventricular systole window: control circuit 206 may determine that oversensing criteria are met at block 610 and the Vsense signal is an oversensed event at block 612. If the motion signal crosses a ventricular systolic event threshold amplitude within a threshold time interval or within a ventricular systole window after the Vsense signal, control circuit 206 may determine that oversensing criteria are not met at block 610 and that the Vsense signal is a valid Vsense signal at block 618.

Additionally or alternatively, control circuit 206 may determine a secondary motion metric by determining a summation of motion signal sample points within the ventricular systole window that follows the received Vsense signal, and/or control circuit 206 may determine a sample point count of sample points within the ventricular systole window that have an amplitude greater than a ventricular systolic event threshold amplitude. The summation metric and/or the sample point count metric determined from the ventricular systole window may be compared to respective threshold values. When the summation metric and/or sample point count metric is/are less than respective threshold values, control circuit 206 may determine that oversensing criteria are met at block 610 and the Vsense signal is an oversensed event at block 612.

In some examples, control circuit 206 may determine a summation metric and/or sample point count metric from the motion signal sensed during the motion metric time interval outside of the ventricular systole window. In this case, control circuit 206 may determine if a ratio of the motion metric determined during the ventricular systole window to the motion metric determined outside the ventricular systole window is less than a threshold value. Control circuit 206 may determine that oversensing criteria are met at block 610 when at least a ratio of a motion metric determined from the motion signal sensed during a ventricular systole window to the motion metric determined from the motion signal sensed outside the ventricular systole window is less than a threshold.

The motion metric determined from the motion signal sensed during the motion metric time interval but outside the ventricular systole window may be determined from a “ventricular diastole window” that follows the ventricular systole and corresponds to the time of the ventricular diastolic phase if the Vsense signal is a true R-wave. For instance, control circuit 206 may determine a ratio of a motion metric determined from the motion signal sensed during a ventricular systole window to a motion metric determined from a ventricular diastole window (immediately following the ventricular systole window). If the ratio is not greater than a threshold ratio indicating greater motion during ventricular systole than during ventricular diastole, the Vsense event may be determined to be an oversensed event at block 612. However, if the motion metric determined during the ventricular diastole window is greater than a threshold value, non-cardiac motion or noise may be contributing to the motion signal such that the motion signal may not be reliable for determining if the Vsense signal is an oversensed event. Control circuit 206 may determine that the motion signal is unreliable for discriminating between oversensed events and true Vsense signals at block 611 and provide a ventricular pacing response at block 614.

In still other examples, control circuit 206 may compare the motion signal waveform morphology during the ventricular systole window to a previously established ventricular systole template morphology. Other motion signal features that may be determined during a portion of the motion metric time interval, e.g., during a ventricular systole window for comparison to secondary oversensing criteria may include a maximum slope, a maximum peak, and/or number of maximum peaks. Control circuit 206 may compare one or more of the foregoing examples of motion signal features or metrics that involve setting a ventricular systole window in any combination to secondary oversensing criteria at block 610 in response to the motion metric(s) determined at block 605 from the motion signal spanning the entire motion metric time interval not meeting oversensing criteria at block 606.

Further disclosed herein is the subject matter of the following examples:

Example 1. A medical device including a motion sensor configured to sense a motion signal and a sensing circuit configured to sense a cardiac electrical signal and sense a cardiac electrical event from the cardiac electrical signal. The medical device may further include a control circuit configured to receive the motion signal sensed by the motion sensor during a motion metric time interval and receive a cardiac electrical event signal during the motion metric time interval, wherein the control circuit receives the cardiac electrical event signal from the sensing circuit when the sensing circuit senses the cardiac electrical event. The control circuit may be further configured to determine a first motion metric from the motion signal sensed by the motion sensor during the motion metric time interval in response to the cardiac electrical event signal being received during the motion metric time interval, determine that oversensing criteria are met based on at least the first motion metric and determine that the cardiac electrical event signal is an oversensed signal in response to the oversensing criteria being met.

Example 2. The medical device of example 1 further including a pulse generator configured to a generate at least one pacing pulse in response to the control circuit determining that the cardiac electrical event signal is an oversensed signal.

Example 3. The medical device of example 2 wherein the pulse generator is further configured to generate the at least one pacing pulse by generating a plurality of pacing pulses at a fixed pacing rate.

Example 4. The medical device of any one of examples 2 or 3 wherein the control circuit is further configured to start a pacing escape interval and withhold restarting the pacing escape interval in response to the cardiac electrical event signal being received during the motion metric time interval.

Example 5. The medical device of example 4, wherein the control circuit is further configured to determine that the pacing escape interval expires and the pulse generator is further configured to generate a first pacing pulse of the at least one pacing pulse in response to the control circuit determining that the pacing escape interval is expired and the cardiac electrical event signal being an oversensed signal.

Example 6. The medical device of example 5 wherein the control circuit is further configured to determine that the pacing escape interval expires after the cardiac electrical event signal is received and before an expiration of the motion metric time interval and delay the first pacing pulse until at least an expiration of the motion metric time interval.

Example 7. The medical device of any one of examples 2-6 wherein the control circuit is further configured to switch a pacing mode in response to determining that the cardiac electrical event signal is an oversensed signal.

Example 8. The medical device of any one of examples 1-7 wherein the control circuit is further configured to adjust at least one cardiac event sensing control parameter in response to determining that the cardiac electrical event signal is an oversensed signal.

Example 9. The medical device of example 8 wherein the control circuit is further configured to adjust the at least one cardiac event sensing control parameter by adjusting a sensitivity setting.

Example 10. The medical device of any one of examples 8 or 9 wherein the control circuit is further configured to adjust the at least one cardiac event sensing control parameter by switching to dual sensor cardiac electrical event signal sensing using both a cardiac electrical signal and the motion signal for sensing cardiac event signals.

Example 11. The medical device of any one of examples 8-10 wherein the control circuit is further configured to adjust the at least one cardiac event sensing control parameter by adjusting a post-atrial ventricular blanking period.

Example 12. The medical device of any one of examples 1-11, wherein the control circuit is further configured to determine the first motion metric by determining a summation of sample point amplitudes of the motion signal sensed during the motion metric time interval and determine that oversensing criteria are met based on at least the first motion metric by determining that the summation of sample point amplitudes is less than a threshold value.

Example 13. The medical device of any one of examples 1-12, wherein the control circuit is further configured to determine the first motion metric by determining a count of sample point amplitudes of the motion signal sensed during the motion metric time interval that are greater than a threshold amplitude and determine that the oversensing criteria are met based on at least the first motion metric by determining that the count of sample point amplitudes is less than a threshold value.

Example 14. The medical device of any of examples 1-13 wherein the control circuit is further configured to determine the first motion metric based on an analysis of sample points during a first portion of the motion metric time interval and determine that the first motion metric is greater than a threshold value. The control circuit may be further configured to determine a second motion metric based on an analysis of sample points during a second portion of the motion metric time interval that is different than the first portion of the motion metric time interval and determine that the oversensing criteria are met based on at least the second motion metric when the first motion metric is greater than the threshold value.

Example 15. The medical device of any one of examples 1-13 wherein the control circuit is further configured to determine the first motion metric based on an analysis of sample points during a first portion of the motion metric time interval, determine that the first motion metric is greater than a threshold value, determine a second motion metric based on an analysis of sample points during a second portion of the motion metric time interval that is different than the first portion of the motion metric time interval and determine that the motion signal is unreliable for determining that the received cardiac electrical event signal is an oversensed event based on at least the second motion metric when the first motion metric is greater than the threshold value. The medical device may further include a pulse generator configured to generate at least one pacing pulse in response to the control circuit determining that the motion signal is unreliable for determining that the received cardiac electrical event signal is an oversensed event.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. A medical device comprising:
a motion sensor configured to sense a motion signal;
a sensing circuit configured to:
sense a cardiac electrical signal; and
sense a cardiac electrical event from the cardiac electrical signal;
a control circuit configured to:
receive the motion signal sensed by the motion sensor during a motion metric time interval;
receive a cardiac electrical event signal during the motion metric time interval, wherein the control circuit receives the cardiac electrical event signal from the sensing circuit when the sensing circuit senses the cardiac electrical event;
determine a first motion metric from the motion signal sensed by the motion sensor during the motion metric time interval in response to the cardiac electrical event signal being received during the motion metric time interval;
determine that oversensing criteria are met based on at least the first motion metric; and
determine that the cardiac electrical event signal is an oversensed signal in response to the oversensing criteria being met; and
a pulse generator configured to a generate at least one pacing pulse in response to the control circuit determining that the cardiac electrical event signal is an oversensed signal.

2. (canceled)

3. The medical device of claim 1 wherein the pulse generator is further configured to generate the at least one pacing pulse by generating a plurality of pacing pulses at a fixed pacing rate.

4. The medical device of claim 1 wherein the control circuit is further configured to:
start a pacing escape interval; and
withhold restarting the pacing escape interval in response to the cardiac electrical event signal being received during the motion metric time interval.

5. The medical device of claim 4, wherein:
the control circuit is further configured to determine that the pacing escape interval expires; and
the pulse generator is further configured to generate a first pacing pulse of the at least one pacing pulse in response to the control circuit determining that the pacing escape interval is expired and the cardiac electrical event signal being an oversensed signal.

6. The medical device of claim 54, wherein the control circuit is further configured to:
determine that the pacing escape interval expires after the cardiac electrical event signal is received and before an expiration of the motion metric time interval; and
delay the first pacing pulse until at least an expiration of the motion metric time interval.

7. The medical device of claim 1 wherein the control circuit is further configured to switch a pacing mode in response to determining that the cardiac electrical event signal is an oversensed signal; and
the pulse generator is further configured to generate the at least one pacing pulse according to the pacing mode.

8. The medical device of claim 1 wherein the control circuit is further configured to adjust at least one cardiac event sensing control parameter in response to determining that the cardiac electrical event signal is an oversensed signal.

9. The medical device of claim 8 wherein the control circuit is further configured to adjust the at least one cardiac event sensing control parameter by adjusting a sensitivity setting.

10. The medical device of claim 8 wherein the control circuit is further configured to adjust the at least one cardiac event sensing control parameter by switching to dual sensor cardiac electrical event signal sensing using both a cardiac electrical signal and the motion signal for sensing cardiac event signals.

11. The medical device of claim 8 wherein the control circuit is further configured to adjust the at least one cardiac event sensing control parameter by adjusting a post-atrial ventricular blanking period.

12. The medical device of claim 1 wherein the control circuit is further configured to:
determine the first motion metric by determining a summation of sample point amplitudes of the motion signal sensed during the motion metric time interval; and
determine that oversensing criteria are met based on at least the first motion metric by determining that the summation of sample point amplitudes is less than a threshold value.

13. The medical device of claim 1 wherein the control circuit is further configured to:
determine the first motion metric by determining a count of sample point amplitudes of the motion signal sensed during the motion metric time interval that are greater than a threshold amplitude; and
determine that the oversensing criteria are met based on at least the first motion metric by determining that the count of sample point amplitudes is less than a threshold value.

14. The medical device of claim 1 wherein the control circuit is further configured to:
determine the first motion metric based on an analysis of sample points during a first portion of the motion metric time interval;
determine that the first motion metric is greater than a threshold value;
determine a second motion metric based on an analysis of sample points during a second portion of the motion metric time interval that is different than the first portion of the motion metric time interval; and
determine that the oversensing criteria are met based on at least the second motion metric when the first motion metric is greater than the threshold value.

15. The medical device of claim 1 wherein the control circuit is further configured to:
determine the first motion metric based on an analysis of sample points during a first portion of the motion metric time interval;
determine that the first motion metric is greater than a threshold value, determine a second motion metric based on an analysis of sample points during a second portion of the motion metric time interval that is different than the first portion of the motion metric time interval; and
determine that the motion signal is unreliable for determining that the received cardiac electrical event signal is an oversensed event based on at least the second motion metric when the first motion metric is greater than the threshold value;
the medical device further comprising a pulse generator configured to generate at least one pacing pulse in response to the control circuit determining that the motion signal is unreliable for determining that the received cardiac electrical event signal is an oversensed event.

16. A non-transitory computer readable storage medium storing instructions that, when executed by a medical device, cause the medical device to:
receive a motion signal during a motion metric time interval;
receive a cardiac electrical signal;
sense a cardiac electrical event from the cardiac electrical signal during the motion metric time interval;
determine a first motion metric from the motion signal received during the motion metric time interval in response to the cardiac electrical event signal being sensed during the motion metric time interval;
determine that oversensing criteria are met based on at least the first motion metric; and
determine that the cardiac electrical event signal is an oversensed signal in response to the oversensing criteria being met.

17. The non-transitory computer readable storage medium of claim 16 wherein the instructions further cause the medical device to:
start a pacing escape interval; and
determine that the pacing escape interval expires after the cardiac electrical event signal is received and before an expiration of the motion metric time interval; and
delay the first pacing pulse until at least an expiration of the motion metric time interval.

18. The non-transitory computer readable storage medium of claim 16 wherein the instructions further cause the medical device to adjust at least one cardiac event sensing control parameter in response to determining that the cardiac electrical event signal is an oversensed signal.

19. The non-transitory computer readable storage medium of claim 16 wherein the instructions further cause the medical device to:
determine the first motion metric by determining a summation of sample point amplitudes of the motion signal sensed during the motion metric time interval; and
determine that oversensing criteria are met based on at least the first motion metric by determining that the summation of sample point amplitudes is less than a threshold value.

20. The non-transitory computer readable storage medium of claim 16 wherein the instructions further cause the medical device to:
determine the first motion metric by determining a count of sample point amplitudes of the motion signal sensed during the motion metric time interval that are greater than a threshold amplitude; and
determine that the oversensing criteria are met based on at least the first motion metric by determining that the count of sample point amplitudes is less than a threshold value.

21. The non-transitory computer readable storage medium of claim 16 wherein the instructions further cause the medical device to:
determine the first motion metric based on an analysis of sample points during a first portion of the motion metric time interval;
determine that the first motion metric is greater than a threshold value;
determine a second motion metric based on an analysis of sample points during a second portion of the motion metric time interval that is different than the first portion of the motion metric time interval; and
determine that the oversensing criteria are met based on at least the second motion metric when the first motion metric is greater than the threshold value
